(19) United States
(12) Patent Application Publication
(10) Pub. No.: US 2016/0250134 A1
(43) Pub. Date: Sep. 1, 2016

(54) POLYMERS AND USES THEREOF

(71) Applicant: REVOLYMER (U.K.) LIMITED, Deeside (GB)

(72) Inventors: Thomas Charles CASTLE, Flintshire (GB); Robert Louis FINCH, Flintshire (GB); David Alan PEARS, Flintshire (GB); Brian David YOUNG, Flintshire (GB)

(73) Assignee: REVOLYMER (U.K.) LIMITED, Deeside (GB)

(21) Appl. No.: 15/050,769

(22) Filed: Feb. 23, 2016

(30) Foreign Application Priority Data
Feb. 27, 2015 (GB) 1503393.9

(51) Int. Cl. A61K 8/81; A61Q 5/06; A61Q 1/10; C08F 22/02; A61Q 19/00

(52) U.S. Cl. CPC A61K 8/8164; C08F 22/02; A61Q 19/00; A61Q 1/10; A61Q 5/06

(57) ABSTRACT

Uses of polymers including monomeric units based on salts of itaconic acid as film formers are disclosed. The polymers confer good film-forming characteristics to the compositions to which they are added, making them suitable for use in cosmetic and personal care product, in particular those that are applied to the skin, hair or nails. The polymers offer a variety of benefits to the products into which they are incorporated, increasing their performance, longevity or adhesion to the human body and improving their feel or appearance during use. Also disclosed are methods of preparing the polymers and the cosmetic and personal care compositions.

The present invention relates to polymers and their use as film formers in cosmetic and personal care compositions, as well as to methods of making the polymers and personal care and cosmetic compositions comprising them. More particularly, the present invention relates to polymers comprising monomeric moieties formed from the polymerisation of itaconic acid salts, and their use in personal care and cosmetic compositions suitable for application to the skin, hair, or nails.

### BACKGROUND OF THE INVENTION

There is an increasing demand for products obtained from raw materials that have been derived from natural or renewable resources. Consumers of many personal care and cosmetic formulations are amongst those most desirous of products that are so derived.

In order to deliver the desired performance that consumers require, modern personal care products are often inherently complex formulations, meaning that it is often not possible to replace all of the ingredients with naturally- or renewably-derived alternatives. Nonetheless, the replacement of at least some of these ingredients with greener options is a possibility. For instance the petrochemically-derived polymers of acrylic acid traditionally used to thicken formulations can in many instances be replaced with a polysaccharide such as xanthan gum (US2013336907).

Film forming agents fulfill a variety of important roles in personal care products ranging from providing an effective barrier on the skin to providing hold in a hair-styling product. In general there is a lack of natural film formers that can assume this role, particularly those that are entirely derived from natural feedstocks.

The present invention was devised with the foregoing in mind.

### SUMMARY OF THE INVENTION

According to a first aspect of the present invention there is provided a cosmetic or personal care composition comprising a polymer, the polymer comprising:
a) 0-100% of monomeric moieties formed from the polymerisation of itaconic acid salts;
b) 0-90% of monomeric moieties formed from the polymerisation of itaconic acid, esters of itaconic acid, amides of itaconic acid and/or itaconic anhydride,
   wherein the quantity of the polymer present within the cosmetic or personal care composition is sufficient to form a film on an external surface of the human body to which the composition is applied.

According to a further aspect of the present invention there is provided a film forming cosmetic or personal care composition comprising a polymer, the polymer comprising:
a) 0-100% of monomeric moieties formed from the polymerisation of itaconic acid salts;
b) 0-90% of monomeric moieties formed from the polymerisation of itaconic acid, esters of itaconic acid, amides of itaconic acid and/or itaconic anhydride,
   wherein the quantity of the polymer present within the cosmetic or personal care composition is sufficient to form a film on an external surface of the human body to which the composition is applied.

According to a further aspect of the present invention there is provided a method for forming a film on an external surface of the human body, the method comprising the step of:
a) applying to the external surface, a polymer as defined herein or a cosmetic or personal care composition as defined herein;
   wherein the amount of polymer or cosmetic or personal care composition applied to the external surface is sufficient to form a film on the external surface.

According to a further aspect of the present invention there is provided a use of a polymer comprising:
a) 10-100% of monomeric moieties formed from the polymerisation of itaconic acid salts;
b) 0-90% of monomeric moieties formed from the polymerisation of itaconic acid, esters of itaconic acid, amides of itaconic acid and/or itaconic anhydride,
   as a film forming agent.

According to a further aspect of the present invention there is provided a cosmetic or personal care composition comprising a polymer as defined herein.

According to a further aspect of the present invention there is provided a process for the preparation of a polymer as defined herein, the process comprising the steps of:
a) providing a monomer mixture comprising water and either:
   i. one or more salts of itaconic acid,
   ii. a base, to which itaconic acid is subsequently added, or
   iii. itaconic acid, to which a base is subsequently added;
b) reacting the mixture of step a) with a polymerisation initiator;
c) isolating the polymeric product resulting from step b).

According to a further aspect of the present invention there is provided a polymer obtainable, obtained or directly obtained by a process as defined herein.

According to a further aspect of the present invention there is provided a process for the preparation of a cosmetic or personal care composition, the process comprising the step of mixing a polymer as defined herein with one or more cosmetically acceptable diluents, excipients or carriers.

According to a further aspect of the present invention there is provided a cosmetic or personal care composition obtainable, obtained or directly obtained by a process as defined herein.

According to a further aspect of the present invention, there is provided a use of a polymer comprising:
a) 10-100% of monomeric moieties formed from the polymerisation of itaconic acid salts;
b) 0-90% of monomeric moieties formed from the polymerisation of itaconic acid, esters of itaconic acid, amides of itaconic acid and/or itaconic anhydride,
   as a film forming agent in a cosmetic or personal care composition

According to a further aspect of the present invention, there is provided, a process for the preparation of a cosmetic or personal care composition as defined herein, the process comprising the steps of:
a) providing a monomer mixture comprising water and either:
   i. one or more salts of itaconic acid,
   ii. a base, to which itaconic acid is subsequently added, or
   iii. itaconic acid, to which a base is subsequently added;
b) reacting the mixture of step a) with a polymerisation initiator;
c) isolating the polymeric product resulting from step b); and
d) mixing the isolated polymeric product of step c) with one or more cosmetically acceptable diluents, excipients or carriers.

### DETAILED DESCRIPTION OF THE INVENTION

### Definitions

In this specification, unless otherwise specified, the terms discussed below will be understood to have the indicated meaning.

The terms “itaconic acid salt”, “salts of itaconic acid” and “X itaconate” wherein X is a suitable counter ion, mentioned herein all synonymously refer to salts formed by a reaction between itaconic acid (or monomeric moieties formed by the polymerisation of itaconic acid) with a base. Bearing in mind that itaconic acid is a dicarboxylic acid, the salt may be the monosalt (in which case only one of the acid functionalities has been neutralised) or the disalt (in which case both of the acid functionalities have been neutralised). The salt may be formed by reaction with a monovalent base (e.g. NaOH), in which case 2 moles of base are required to fully neutralise 1 mole of acid, or the salt may be formed from a divalent base (e.g. Mg(OH)₂), in which case 1 mole of base is required to fully neutralise 1 mole of acid. It will equally be understood that where the salt is present as the monosalt, the remaining acid functionality may be derivitised by means of an esterification or amidation reaction. In this regard, salts discussed herein will be understood to include monoester itaconate salts and monoamide itaconate salts.

The terms “itaconate ester” and “ester(s) of itaconic acid” synonymously refer to esters formed by the esterification of itaconic acid, or monomeric moieties formed by the polymerisation of itaconic acid. The ester may be present as the mono ester (in which case the other acid functionality may have optionally been derivatised by an amidation reaction), or a diester (in which case the two ester groups may be identical or different). Unless otherwise specified, the ester is not a salt.

The terms “amides of itaconic acid” and “amido itaconate” synonymously refer to amides formed by the amidation of itaconic acid, or monomeric moieties formed by the polymerisation of itaconic acid. The amide may be present as the monoamide (in which case the other acid functionality may have optionally been derivatised by an esterification reaction), or a diamide (in which case the two amido groups may be identical or different). It will be understood that primary, secondary and tertiary amides are included. Unless otherwise specified, the amide is not a salt.

The terms “natural” or “naturally-derived” synonymously refer to a product that is ultimately derived from a feedstock that has been produced from a non-petrochemical source. Examples include extraction from suitable plants, fungi and bacteria, as well as fermentation techniques. Following extraction of the raw material the product or isolate may then be modified further via chemical or other processing to produce the itaconic acid.

The term “polymer” encompasses linear and branched polymers.

The term “homopolymer” refers to a polymer composed of plurality of identical monomeric moieties.

The term “copolymer” refers to a polymer composed of two or more different monomeric moieties. The copolymer may be an alternating copolymer, a block copolymer, a random copolymer or a graft copolymer.

The term “alkyl” encompasses linear or branched alkyl group of 1 to 20 carbon atoms, suitably 1 to 10 carbon atoms, more suitably 1 to 5 carbon atoms. Exemplary alkyl groups include methyl groups, ethyl groups, isopropyl groups, n-propyl groups, butyl groups, tert-butyl groups and pentyl groups.

### Methods of Forming Films

As described hereinbefore, the present invention provides a method for forming a film on an external surface of the human body, the method comprising the step of:
a) applying to the external surface, a polymer as defined herein or a cosmetic or personal care composition as defined herein;
   wherein the amount of polymer or cosmetic or personal care composition applied to the external surface is sufficient to form a film on the external surface.

It has surprisingly been found that polymers defined herein comprising monomeric moieties derived from the polymerisation of salts of itaconic acid exhibit remarkable film-forming properties, making them well-suited for use as film formers on a variety of different external bodily surfaces including keratin based substrates such as skin (including the lips), nails and hair.

Film forming agents (also known as film formers) are a class of substances capable of forming a film upon application to a solid surface. Film formers are essential components within a range of products, ranging from paints, coating and lacquers to cosmetic and personal care compositions. When applied to keratinous material like skin hair or nail they tend to leave a pliable, cohesive and continuous covering film or coating. The film left by film forming agents ensures that a personal care product is distributed evenly guaranteeing that any cosmetic or functional benefits are homogenous. Film forming materials can change the mechanical features of the keratin, for instance by soothing features of the skin (e.g. wrinkles) or retaining the shape or texture of hair. They may alter the skin feel or texture of such products, and can help prevent transepidermal water loss, meaning that they can be used to increase the substantivity (i.e. the length of time it is retained) of an active ingredient or increase the resistance of the product to sweat, sebum and/or water. Polymers are a key class of film forming material in personal care products.

Itaconic acid (otherwise known as methylenesuccinic acid) is an unsaturated dicarboxylic acid with an empirical formula of C₅H₆O₄. Itaconic acid is produced industrially by the fermentation of carbohydrates including corn starch and glucose using Aspergillus terreus. Itaconic acid can also be obtained through the distillation of citric acid. The structure of itaconic acid may be represented by formula (A) or formula (B) shown below:

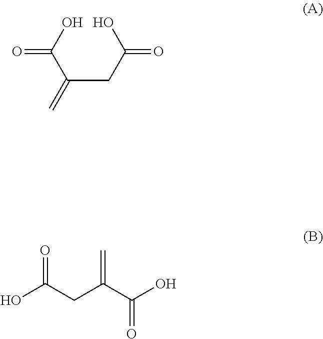

The polymers defined herein may be prepared by polymerising itaconic acid and/or esters and/or amides thereof, followed by partial or full neutralisation with one or more base. Suitably, polymers defined herein are suitably prepared by polymerisation of a preformed itaconate salt. For instance itaconic acid may be neutralised with a monovalent inorganic base such as sodium hydroxide or potassium hydroxide to yield sodium itaconate or potassium itaconate respectively. Alternatively, organic bases including amines or ammonium hydroxide may be used to neutralise the carboxylic functionality of the above mentioned monomers. Neutralisation can be a highly exothermic reaction so neutralisation will normally be carried out at a temperature between 1° C. and 20° C.

Itaconic acid contains dicarboxylic acid functionality and therefore two molar equivalents of a monovalent inorganic base are required to completely neutralise the carboxylic acid functionality. Partial neutralisation is considered to be anything greater than 0% but less than 100% neutralisation of carboxylic acid functionality. The structure of sodium itaconate (100% neutralisation) is represented by formula (C) shown below, whereas the structure of potassium itaconate (50% neutralisation) is represented by formula (D) shown below:

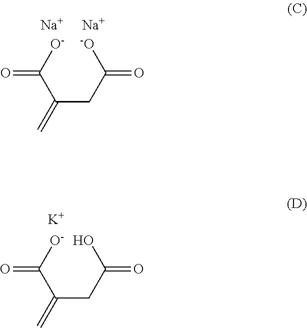

Where the polymer includes individual monomeric moieties that are 50% neutralised (as in the case of the polymerised equivalent of formula (D) shown above), and bearing in mind the asymmetry of itaconic acid about the polymerisable C═C double bond, it will be appreciated that either of the acid functionalities may have been neutralised.

Divalent metal salts of itaconic acid may also be prepared. For example magnesium, calcium or zinc salts of itaconic acid. One mole of magnesium hydroxide, calcium hydroxide or zinc hydroxide is required to completely neutralise one mole of itaconic acid. Similarly, it will be understood that polymers formed from such salts may be such that i) both acid functionalities of a single monomeric moiety are ionically associated with a divalent counter ion (e.g. Mg²⁺), or ii) one of the acid functionalities of one monomeric moiety and one of the acid functionalities of an adjacent monomeric moiety are ionically associated with a divalent counter ion (e.g. Mg²⁺).

In an embodiment, the polymers defined herein are used as film-forming agents in a cosmetic or personal care product. As a consequence of their film forming properties, the polymers defined herein find uses in a variety of cosmetic and personal care products. In particular, the polymers defined herein may be used in any cosmetic and personal care product in which a film former is traditionally used. When used as film formers, the polymers defined herein confer a variety of benefits to the products into which they are incorporated, increasing their performance, longevity or adhesion to the human body and improving the feel or appearance during use. In particular, when used in skin care products, the polymers defined herein serve to facilitate the application of an even layer of the product to the skin. Similarly, the polymers may equally function to increase moisture levels in the skin (i.e. act as an emollient) and can help to increase the barrier performance of products applied topically. In hair styling products, the polymers serve as fixatives in order to allow the hair to maintain an artificial style or shape, whilst also enhancing the appearance of the hair, for instance by increasing its shine or adding lustre. In products applied to the nails (such as nail varnish) the polymers may comprise the primary basis of the film on the nails. A further advantage of the polymers defined herein is that itaconic acid can be derived from a natural source, thereby contributing to the preparation of comparatively greener consumer products.

It will be understood that the cosmetic or personal care product does not encompass oral care products (i.e. products designed for use in or around the oral cavity). Oral care products will be understood as being distinct from make-up products that are applied to the lips (e.g. lip stick and lip balm). Hence, it will be understood that the external surface of the human body does not include those surfaces present within the oral cavity.

When used as a film former in a personal care product the polymers may be used to impart any benefit that may be achieved by the use of a film former known in the art. Non-limiting examples include styling, holding, providing length, curling, building, and separating hair on the head, eyelashes or elsewhere on the human body; providing the retention of active ingredients to the skin including for instance UV filters, emollients/moisturizers, antioxidants, treatments for aged skin and photoaging, rosacea, acne, and other skin concerns; assisting in the spread or application of products on the skin or hair; modifying the feel, texture and organoleptic profile of products; and increasing the resistance of the product to sweat, sebum and environmental factors such as exposure to water. In preferred embodiments the polymers provide styling for the hair that has enhanced environmental resistance (e.g. to humidity and temperature) or prevent transepidermal water loss and provide moisturisation on the skin. In another preferred embodiment the polymers provide a stretching effect on the skin to minimise the visible appearance of defects on the skin including folds, ridges or creases such as wrinkles, fine lines and crow's feet.

In an embodiment, the itaconic acid salts are group 1-12 metal salts or quaternary ammonium salts. Exemplary group 1-12 metal salts include alkaline metal salts, alkaline earth metal salts and transition metal salts. Quaternary ammonium salts include those in which the quaternary ammonium cation has the formula NR₄⁺, in which each R group is independently selected from H or (1-6C)alkyl. Suitably, the itaconic acid salts are selected from sodium salts, potassium salts, calcium salts, magnesium salts, iron salts and zinc salts. More suitably, the itaconic acid salts are selected from sodium salts and potassium salts.

In another embodiment, the monomeric moieties formed from the polymerisation of itaconic acid salts each independently have a structure according to formula (I) shown below:

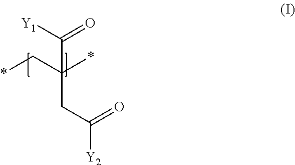

wherein
Y₁ is selected from:
   (i) a group O⁻X₁, wherein X₁ is a monovalent or divalent positively charged atom or group,
   (ii) a group O—R₁, and
   (iii) a group —N(Rₐ)(R_{b}),
      wherein R₁, Rₐ and R_{b} are each independently selected from hydrogen and (1-6C)alkyl;
Y₂ is selected from:
   (i) a group O⁻X₂, wherein X₂ is a monovalent or divalent positively charged atom or group,
   (ii) a group O—R₂, and
   (iii) a group —N(R_{c})(R_{d}),
      wherein R₂, R_{c} and R_{d} are each independently selected from hydrogen and (1-6C)alkyl,
and the monomeric moieties formed from the polymerisation of itaconic acid, esters of itaconic acid and/or amides of itaconic acid each independently have a structure according to formula (II) shown below:

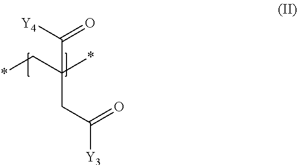

wherein
Y₃ is selected from:
   (i) a group O—R₃, and
   (ii) a group —N(Rₑ)(R_{f}),
      wherein R₃, Rₑ and R_{f} are each independently selected from hydrogen and (1-6C)alkyl; and
Y₄ is selected from:
   (i) a group O—R₄, and
   (ii) a group —N(R_{g})(Rₕ),
      wherein R₄, R_{g} and Rₕ are each independently selected from hydrogen and (1-6C)alkyl
with the proviso that at least one or Y₁ and Y₂ is a group O⁻X₁ or O⁻X₂.

In another embodiment, X₁ and X₂ are each independently selected from group 1-12 atoms and quaternary ammonium ions. Suitably, the group 1-12 atoms are selected from Na, K, Mg, Ca, Fe and Zn, More suitably, the group 1-12 atoms are selected from Na and K. Most suitably, the group 1-12 atoms are Na.

In another embodiment, R₁, R₂, R₃ and R₄ are each independently selected from hydrogen and (1-4C)alkyl. Suitably, R₁, R₂, R₃ and R₄ are each independently selected from hydrogen and (1-2C)alkyl.

In another embodiment, Rₐ, R_{b}, R_{c}, R_{d}, Rₑ, R_{f}, R_{g} and Rₕ are each independently selected from hydrogen and (1-4C)alkyl. Suitably, Rₐ, R_{b}, R_{c}, R_{d}, Rₑ, R_{f}, R_{g} and Rₕ are each independently selected from hydrogen and (1-2C)alkyl.

In another embodiment, Y₁ is a group O⁻X₁ and Y₂ is a group O—R₂, wherein X₁ is as defined in formula (I) and R₂ is hydrogen.

In another embodiment, Y₂ is a group O⁻X₂ and Y₁ is a group O—R₁, wherein X₂ is as defined in formula (I) and R₁ is hydrogen.

In another embodiment, Y₁ is a group O⁻X₁ and Y₂ is a group O⁻X₂, wherein both X₁ and X₂ are as defined in formula (I).

In another embodiment, the monomeric moieties formed from the polymerisation of itaconic acid salts each independently have a structure according to formula (I) shown below:

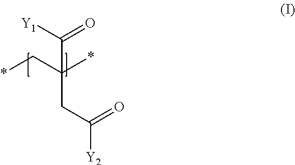

wherein
Y₁ is selected from:
   (i) a group O⁻X₁,
   (ii) a group O—R₁, wherein R₁ is hydrogen;
Y₂ is selected from:
   (i) a group O⁻X₂,
   (ii) a group O—R₂, wherein R₂ is hydrogen,
and the monomeric moieties formed from the polymerisation of itaconic acid and/or esters of itaconic acid each independently have a structure according to formula (II) shown below:

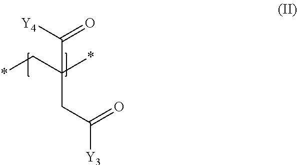

wherein
Y₃ is a group O—R₃, and
Y₄ is a group O—R₄;
with the proviso that at least one or Y₁ and Y₂ is a group O⁻X₁ or O⁻X₂.

In another embodiment, the polymer comprises, consists essentially of, or consists of monomeric moieties formed from the polymerisation of one or more of itaconic acid, sodium itaconate, potassium itaconate, ammonium itaconate, itaconic acid monomethyl ester, itaconic acid dimethyl ester, itaconic acid monoethyl ester and itaconic acid diethyl ester. Suitably, the polymer comprises, consists essentially of, or consists of monomeric moieties formed from the polymerisation of one or more of itaconic acid, sodium itaconate, potassium itaconate, itaconic acid monoethyl ester and itaconic acid diethyl ester. More suitably, the polymer comprises, consists essentially of, or consists of monomeric moieties formed from the polymerisation of one or more of itaconic acid, sodium itaconate and itaconic acid monoethyl ester. Even more suitably, the polymer comprises, consists essentially of, or consists of monomeric moieties formed from the polymerisation of one or more of itaconic acid, sodium itaconate.

In another embodiment, the polymer comprises 25-100% of monomeric moieties formed from the polymerisation of itaconic acid salts. Suitably, the itaconic acid salts are sodium itaconate.

In another embodiment, the polymer comprises 50-100% of monomeric moieties formed from the polymerisation of itaconic acid salts. Suitably, the itaconic acid salts are sodium itaconate.

In another embodiment, the polymer comprises 75-100% of monomeric moieties formed from the polymerisation of itaconic acid salts. Suitably, the itaconic acid salts are sodium itaconate.

In another embodiment, the polymer comprises 90-100% of monomeric moieties formed from the polymerisation of itaconic acid salts. Suitably, the itaconic acid salts are sodium itaconate.

In a particularly suitable embodiment, the polymer is:
a) a homopolymer formed from the polymerisation of sodium itaconate, or
b) a copolymer formed from the polymerisation of sodium itaconate and itaconic acid.
   It will be understood that depending on the degree of neutralisation of the itaconic acid salt starting materials, the resulting polymer may be one in which all of the monomeric moieties have been partially or fully neutralised (in which case the polymer is a homopolymer formed from sodium itaconate monomeric moieties), or one in which only some of the monomeric moieties have been partially or fully neutralised (in which case the polymer is a copolymer formed from sodium itaconate monomeric moieties and un-neutralised itaconic acid monomeric moieties). It will be understood that homopolymers formed from the polymerisation of sodium itaconate include polymers formed from a mixture of monosalt and disalt sodium itaconate monomeric moieties.

In another embodiment, the polymer has a degree of neutralisation of 50-100%. Suitably, the polymer has a degree of neutralisation of 60-70%.

In another embodiment, the polymer is a copolymer that has been prepared by adjusting the pH of a batch of itaconic acid such that less than 50% of the acidic monomers have been neutralised.

In another embodiment, the polymer has a molecular weight of 4000-20000 Da. Suitably, the polymer has a molecular weight of 8000-12000 Da.

In another embodiment, the polymer has a molecular weight of 500-50000 Da. Suitably, the polymer has a molecular weight of 2000-50000 Da. More suitably, polymer has a molecular weight of 4000-50000 Da.

Suitably, the polymer is water soluble or is dispersible in water.

In another embodiment, the cosmetic or personal care composition is for application to the skin, hair, or nails, in which case the external surface is skin (including the lips), hair or nails.

In the instance of a composition for use on the hair, the cosmetic composition can be a shampoo, conditioner or hair styling product. Most suitably, the composition is a styling agent such as a hair spray, hair mousse, hair gel, hair wax, hair pomade, hair clay, hair volumiser and putty.

In the instance of a composition for use on the skin, the cosmetic composition can be a leave-on skin care product, a rinse-off product or a make-up/colour cosmetic product. Leave-on skin care products include those for application to the face and body. Suitable compositions include those for protecting, treating and changing the appearance of the skin. These include products that are used to increase and maintain levels of moisture in the skin or repair dry skin, such as moisturising or barrier creams for use on the face and body and hand cream. The skin care product could be one within the area of sun care, either containing one or more UV filters designed to protect the wearer from the negative effects of exposure to sun, an after sun designed to help repair or treat skin that has been exposed to the sun, or a self-tanning product containing an active ingredient that causes a change in colour in the skin without exposure to the sun. Further skin care products include those for treating skin conditions, such as acne and blemishes. The cosmetic composition can include attributes or both a make-up and skin care product, for example a concealer product. Particularly suitable cosmetic compositions include multifunctional products that include a number of benefits, including for instance moisturisation, UV protection and some of the benefits associated with a foundation product. These are sold under a variety of names by marketers of personal care product in order to convey their multifunctional benefits, currently including for instance AA, BB, CC, DD and EE creams. Examples of suitable rinse-off skin care products include shower gel, bath foam and similar products. Examples of suitable colour cosmetic products for use on the skin include lip, face and eye products. The lip products include for example, lipstick, lip gloss, lip liner, lip plumper, lip balm, lip sheer, lip ink, lip conditioner, lip primer or lip booster. The face make-up products include for example, foundation, face powder, concealer, blusher and bronzer. The eye make-up products include for example, eye shadow, eyeliner or mascara.

In terms of compositions for application to the nails, a particularly suitable composition is nail polish (also known as nail lacquer) including uncoloured base coat and coloured top coat products that cure on drying, as well as gel coat and similar products that cure on exposure to UV light. The products may include agents for treating conditions present on the nails such as discolouration or fungal infection.

In an embodiment, the cosmetic or personal care product does not include fluoride ions.

In another embodiment, the cosmetic or personal care product does not include stannous and/or zinc ions.

### Methods of Preparing Polymers

As described hereinbefore, the present invention also provides a process for the preparation of a polymer as defined herein, the process comprising the steps of:
a) providing a monomer mixture comprising water and either:
   i. one or more salts of itaconic acid,
   ii. a base, to which itaconic acid is subsequently added, or
   iii. itaconic acid, to which a base is subsequently added;
b) reacting the mixture of step a) with a polymerisation initiator;
c) isolating the polymeric product resulting from step b).

Polymers defined herein having properties that make them attractive ingredients in cosmetic and personal care compositions are prepared according to the method described above.

Given the reactivity of itaconic acid the polymers are synthesised by a free radical polymerisation process. In an embodiment, step b) comprises reacting the mixture of step a) with a polymerisation initiator at a temperature of 50-100° C. A number of free radical initiators are available commercially. Suitable water soluble free radical initiators may include persulfate or peroxide initiators. Persulfate free radical initiators include sodium persulfate, potassium persulfate and ammonium persulfate. Peroxide free radical initiators include hydrogen peroxide and t-butyl hydroperoxide. The half-life of free radical initiators is dependent on a number of factors including temperature and solution pH. Accordingly, the reaction conditions and initiator should be chosen to match each other. Suitably, the polymerisation initiator is selection from hydrogen peroxide or a persulfate initiator (e.g. sodium persulfate)

The polymerisation initiators may be used in solid form, or may be dissolved in a suitable solvent before being added to the mixture resulting from step a). In an embodiment, the suitable solvent is water.

The polymerisation initiators may be used at a concentration of 1 to 50 w/w % with respect to the weight of monomer(s). The choice of level of initiator is dictated by the identity of the initiator, as well as other reaction conditions such as monomer identity, concentration of the reaction. In a suitable embodiment, a persulfate initiator is used at a concentration of between 1.8 and 9.0 w/w %, more suitably 5.0 to 7.0 w/w %. In another embodiment, hydrogen peroxide is used at a concentration between 10.0 and 30.0 w/w %, more suitably 20.0 to 30.0 w/w %.

In an embodiment, step b) comprises the initial step of adjusting the temperature of the mixture resulting from step a) to a temperature of 50-100° C. prior to the addition of the polymerisation initiator. In an alternative embodiment, step b) comprises adding the polymerisation initiator to the mixture resulting from step a), and then adjusting the temperature of the resulting solution to a temperature of 50-100° C. Most suitably, step b) comprises the initial step of adjusting the temperature of the mixture resulting from step a) to a temperature of 60-85° C. prior to the addition of the polymerisation initiator.

In an embodiment, step b) comprises adjusting the temperature of the reaction mixture to 60-100° C. Suitably, step b) comprises adjusting the temperature of the reaction mixture to 60-85° C. Suitably, step b) comprises adjusting the temperature of the reaction mixture to 65-80° C. It will be understood that the term reaction mixture refers to either i) the mixture resulting directly from step a), or ii) the mixture resulting from step a) to which one or more polymerisation initiators has been added as part of step b).

In another embodiment, in step b), the polymerisation initiator may be added in a staggered manner, for example in a two-stage process. Optionally, the temperature of the reaction mixture may be adjusted before, between and/or after additions of polymerisation initiator. In an embodiment, step b) comprises steps of)

(1) adding to the mixture resulting from step a) a quantity of polymerisation initiator

(2) adjusting the temperature of the mixture resulting directly from step (1),

(3) adding to the mixture resulting from step (2) a quantity of polymerisation initiator,

(4) adjusting the temperature of the mixture resulting directly from step (3).

Suitably, step (2) comprises adjusting the temperature to 60-80° C. and step (4) comprises adjusting the temperature to 85-105° C.

Where step b) includes a step of adjusting the temperature of the reaction mixture after the polymerisation initiator has been added, this step may take the form of an excess monomer burnout step.

In an embodiment, the total quantity of polymerisation initiator is added in step b) over a period of 30 minutes to 4 hours. Suitably, the polymerisation initiator is continuously feed to the reaction mixture as an aqueous solution during this period of time.

In another embodiment, step b) comprises reacting the mixture of step a) with a polymerisation initiator for a period of 2 to 96 hours. Suitably, step b) comprises reacting the mixture of step a) with a polymerisation initiator for a period of 4 to 40 hours. More suitably, step b) comprises reacting the mixture of step a) with a polymerisation initiator for a period of 4 to 8 hours. Even more suitably, step b) comprises reacting the mixture of step a) with a polymerisation initiator for a period of 6 to 8 hours.

In an embodiment, in step a), the reaction concentration of the monomers present within the monomer mixture is 5-60 w/w %. In a suitable embodiment, the concentration of the monomers present within the monomer mixture is 40-60 w/w %.

Having regard to step c), the polymers may be isolated by any of the techniques known in the prior art. By way of example, this includes using any known evaporation process including for instance, falling film evaporation, convection and radiation assisted drying, spray drying, freeze drying, drum drying, rotary evaporation and natural drying. Optionally, the polymer can be isolated by some chemical method such as combining it with a solvent in which it has poor or limited solubility (commonly referred to as a ‘non-solvent’) or increasing the ionic strength of the system in order to precipitate or separate it from the solution. As an alternative, the polymer can be left in the solvent used in the manufacturing process and formulated directly into the cosmetic product. Optionally various ingredients can be added to the solution to precipitate the product. Suitably, in step c), the polymer is isolated by diluting the mixture resulting from step b) in deionized water and/or cooling.

### Cosmetic and Personal Care Compositions

As described hereinbefore, the present invention also provides a cosmetic or personal care composition comprising a polymer, the polymer comprising:
a) 0-100% of monomeric moieties formed from the polymerisation of itaconic acid salts;
b) 0-90% of monomeric moieties formed from the polymerisation of itaconic acid, esters of itaconic acid, amides of itaconic acid and/or itaconic anhydride,
   wherein the quantity of the polymer present within the cosmetic or personal care composition is sufficient to form a film on an external surface of the human body to which the product is applied.

Given the remarkable film-forming properties of the polymers defined herein, they find uses in a wide variety of cosmetic and personal care products for application to a range of different external surfaces of the human body, including keratin based substrates such as skin (including the lips), nails and hair. As mentioned hereinbefore, the polymers defined herein may be used in any cosmetic and personal care product in which a film former is traditionally used. When used as film formers, the polymers defined herein confer a variety of benefits to the products into which are incorporated, increasing their performance, longevity or adhesion to the human body and improving the feel or appearance during use. Yet a further advantage of the cosmetic and personal care products of the invention is that itaconic acid can be derived from a natural source, resulting in a comparatively greener end product.

It is important when producing cosmetic compositions that they should be produced using materials that are safe and comply with the regulatory and legal requirements in the markets they are to be sold into. Cosmetic materials are classified using the International Nomenclature of Cosmetic Ingredients (INCI) system. The INCI system (as of February 2015, administered by the Personal Care Products Council) is designed to make easy identification of ingredients used in personal care and cosmetic products. It will be understood by those skilled in the art that generally speaking it will be possible to replace a material with one from a different manufacturer with an identical INCI name without notably changing the resulting product.

A number of ingredients may be used in the cosmetic composition alongside the polymers defined herein in order to create a product that is useful to the consumer. Representative examples of ingredients can be found in the International Cosmetic Ingredient Dictionary and Handbook, Fifteenth Edition, edited by Gottschalck, and Bailey, 2014, the infobase of the Personal Care Products Council, and the Inventory and Common Nomenclature of Ingredients Employed in Cosmetic Products (dated 9 Feb. 2006). These may include for instance, ingredients to create the structure of a product, those that have a non-visually observable benefit to consumers such as active ingredients and those that provide a clear cosmetic benefit such as a pigment.

A number of ingredients that may optionally be selected for use as part of the cosmetic compositions of the invention. It will be appreciated to those skilled in the art that not all of the ingredients will be appropriate to all formulations and that selection of the most appropriate ingredients will depend on selecting the correct properties that are needed in the final product. In many instances many of the most useful ingredients listed as serving one function many be used in another function and such use is within the scope of the invention. For instance many viscosity modifiers may possess some film forming performance and vice-versa.

In an embodiment, the cosmetic or personal care composition may comprise one or more waxes. Suitable waxes include, but are not limited to, those derived from vegetable, animal, petroleum, synthetic or mineral sources. Preferred examples include ozokerite wax, microcrystalline wax, beeswax, carnauba wax, lanolin, rhus succedanea fruit wax, ethoxylated beeswax and candelila wax.

In an embodiment, the cosmetic or personal care composition may comprise one or more oils, fats and/or waxes. Suitable oils, fats and waxes include, but are not limited to, those derived from vegetable, animal, petroleum, synthetic or mineral sources. Preferred examples include castor, olive, jojoba, coconut, sesame, safflower, orange, mineral, canola and various silicone/methicone oils, lanolin and petrolatum. Preferably, for lip products, the cosmetically acceptable diluent, excipient or carrier is selected from an oil, a fat and a wax, or most preferably a mixture thereof. Most lip products (with the exception of lip stain) use mixtures based on oils, fats and/or waxes sometimes referred to as structuring agents or ingredients

Most lip products (with the exception of lip stain) use mixtures based on oils, fats and/or waxes sometimes referred to as structuring agents or ingredients

In an embodiment, the cosmetic or personal care composition may comprise one or more emulsifiers and/or structural agents. The skilled person will appreciate that the function of an emulsifier is entirely different to that of a film former. Suitable emulsifiers or structural agents include cetearyl alcohol, cetyl alcohol, stearyl alcohol, palmitic and stearic acid, polyethylene glycol ether made from stearic or oleic acids such as oleth-30 and steareth-21, Ceteth-10 Phosphate, PEG Castor Oils and PEG Hydrogenated Castor Oils.

In addition to the polymers defined herein, the cosmetic or personal care composition may optionally include one or more further film forming polymers. Examples of suitable film forming polymers include homo and copolymers of styrene, butadiene, ethylene, propylene, isobutylene, methyl vinyl ether, vinyltoluene, vinyl propionate, vinyl alcohol, acrylonitrile, chloroprene, methacrylamidopropyltrimethyl-ammonium chloride, vinyl acetate, urethanes, isoprene, isobutene, vinyl ether, vinylpyrrolidone, vinylimidazole, vinyl neodeconoate, vinylcaprolactam, and esters or salts or amides of acrylic acid, methacrylic acid, maleic acid, crotonic acid and itaconic acid. Non-limiting preferred examples include poly vinylpyrrolidone, copolymers of vinylpyrrolidone and vinyl acetate, copolymers of vinyl acetate crotonates and vinyl neodeconoate, copolymers of vinylcaprolactam with vinylpyrrolidone and dimethylaminoethyl methacrylate, copolymers of vinylpyrrolidone and quaternized vinylimidazole, triacontanyl vinylpyrrolidone, vinylpyrrolidone/DMAPA acrylates copolymer, copolymers of vinylpyrrolidone and dimethylaminoethylmethacrylate, copolymers of octylacrylamide with acrylates and butylaminoethyl methacrylate, copolymers of methyl vinyl ether and maleic anhydride, copolymers of the ethyl, isopropyl and butyl ester of maleic anhydride and methyl vinyl ether, copolymers of the sodium, potassium, calcium or mixed salt of maleic anhydride and methyl vinyl ether, copolymers of isobutylene with ethylmaleimide and hydroxyethyl maleimide, isobutylene/dimethylaminopropyl maleimide/ethoxylated maleimide/maleic acid copolymer, copolymers of methacrylamidopropyltrimethyl-ammonium chloride and vinylpyrrolidone, copolymers of acrylates and octylacrylamide, copolymers of vinylpyrrolidone, acrylate monomers and lauryl methacrylate, copolymers of octylacrylamide with acrylates and butylaminoethyl methacrylate copolymer, copolymers of polyurethanes and monomers such as acrylic acid or aminomethyl propanol-acrylates, copolymers of acrylates and ceteareth-20 methacrylate, copolymers of styrene and acrylate monomers and sodium polystyrene sulfonate,

The optional extra film forming polymer may comprise a naturally derived or modified naturally derived material such as a monosaccharide, disaccharide, oligosaccharide or polysaccharide, such as for instance hydroxyethylcellulose, hydroxypropylcellulose, dehydroxanthan gum, copolymers maltodextrin with vinyl pyrillidone, tapioca starch or hydroxypropyl starch phosphate.

The performance of the itaconate salt polymer and/or optional extra film forming polymers in generating films can be enhanced by other ingredients, for instance wetting agents to ensure they give continuous coverage, adhesion enhancing agents to further increase their longevity on substrates and plasticisers to ensure films remain pliable.

Suitable solvents for inclusion within the cosmetic or personal care composition include water, ethanol, propylene glycol, butylene glycol, isopropyl myristate butane, propane, isobutene volatile silicones such as cyclomethicone; polymeric silicones such as dimethicone; alkylated derivatives of polymeric silicones, such as cetyl dimethicone and lauryl trimethicone; hydroxylated derivatives of polymeric silicones, such as dimethiconol; and mixtures thereof

Suitable clays and organoclays for inclusion within the cosmetic or personal care composition include, but are not limited to, the reaction products of bentonite clays and quaternium ammonium salts, kaolin, hectorite clays and quaternium ammonium salts, or montmorillonite clays and quaternium ammonium salts.

Suitable viscosity modifiers for inclusion within the cosmetic or personal care composition may include carbomer, acrylic acidNP crosspolymer, xanthan gum, sodium chloride, derivatives of cellulose such as hydroxyethyl cellulose, modified starches such as corn starch.

Suitable conditioning agents for inclusion within the cosmetic or personal care composition include, but are not limited to, phenyl trimethicone polyquaternium-59, PEG-12 dimethicone bis-PEG/PPG-20/20 dimethicone, amine functionalised silicones such as amodimethicones such as hydrolysed proteins such as soy, rice and wheat.

Surfactants suitable for inclusion within the cosmetic or personal care composition (especially those for cleansing applications) can be anionic, cationic, non-ionic or amphoteric. Suitable anionic surfactants include the alkyl sulphates, alkyl ether sulphates, alkaryl sulphonates, alkanoyl isethionates, alkyl succinates, alkyl sulphosuccinates, Nalkoyl sarcosinates, alkyl phosphates, alkyl ether phosphates, alkyl ether carboxylates, and alpha-olefin sulphonates, especially their sodium, magnesium ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from one to 10 ethylene oxide or propylene oxide units per molecule, and preferably contain 2 to 3 ethylene oxide units per molecule. Examples include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, disodium laureth sulfosuccinate, disodium laureth-3 sulfosuccinates, dioctyl sodium sulfosuccinate, and combinations thereof.

Nonionic surfactants suitable for inclusion within the cosmetic or personal care composition may include condensation products of aliphatic (C8-C18) primary or secondary linear or branched chain alcohols or phenols with alkylene oxides, usually ethylene oxide and generally having from 6 to 30 ethylene oxide groups. Other suitable nonionics include mono- or di-alkyl alkanolamides, such as coco mono- or di-ethanolamide and coco mono-isopropanolamide.

Amphoteric and zwitterionic surfactants suitable for inclusion within the cosmetic or personal care composition may include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates alkyl amidopropyl hydroxysultaines, acyl taurates and acyl glutamates, wherein the alkyl and acyl groups have from 8 to 19 carbon atoms. Examples include lauryl amine oxide, cocodimethyl sulphopropyl betaine and preferably lauryl betaine, cocamidopropyl betaine and sodium cocamphopropionate.

Examples of suitable cationic surfactants suitable for inclusion within the cosmetic or personal care composition include quaternary ammonium compounds, particularly trimethyl quaternary compounds. Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen, (e. g. bromide), acetate, citrate, lactate, glycolate, phosphate nitrate, sulphate, or alkylsulphate.

The cosmetic or personal care compositions of the present invention may include other ingredients (including active pharmaceutical ingredients) such as those that improve or eradicate age spots, keratoses and wrinkles; analgesics; anesthetics; antiacne agents; antibacterials; antiyeast agents; antifungal agents; antiviral agents; antidandruff agents; antidermatitis agents; antipruritic agents; antiemetics; antimotion sickness agents; antiinflammatory agents; antihyperkeratolytic agents; antidryskin agents; antiperspirants; antipsoriatic agents; antieborrheic agents; hair conditioners and hair treatment agents; antiaging antiwrinkle agents; antiasthmatic agents and bronchodilators; sunscreen agents; antihistamine agents; skin lightening agents; depigmenting agents; vitamins; corticosteroids; tanning agents; hormones; retinoids; topical cardiovascular agents; clotrimazole; ketoconazole; miconazole; griseofulvin; hydroxyzine; diphenhydramine; pramoxine; lidocaine; procaine; mepivacaine; monobenzone; erythidocaine; procaine; mepivacaine; monobenzone; erythromycin; tetracycline; clindamycin; meclocyline; hydroquinone; minocycline; naproxen; ibuprofen; theophylline; cromolyn; albuterol; retinoic acid; 13-cis retinoic acid; hydrocortisone; hydrocortisone 21-acetate; hydro-cortisone 17-valerate; hydrocortisone 17-butyrate; betamethasone valerate; betamethasone dipropionate; triamcinolone acetonide; fluocinonide; clobetasol propionate; benzoyl peroxide; crotamiton; propranolol; promethazine; vitamin A palmitate; vitamin E acetate and mixtures thereof.

In the case of a hair spray product the cosmetic composition can be delivered either from an aerosol (delivered from a pressurised container) or from a pump spray bottle. This may necessitate a difference in the type of diluent, excipient or carrier present in the product. For instance in products that are delivered by an aerosol format the diluent, excipient or carrier will tend to be a volatile material that can act as a propellent.

Suitably, the cosmetic compositions of the invention may further comprise one or more of an emollient, a colorant, a moisturizer, a UV blocker, an active agent, an antioxidant, a vitamin, a lip plumping agent (for lip products only), a fragrance, a flavour or flavourant, a sweetening agent, a vegetable or herb extract and/or a preservative. The pH of the formulation may be adjusted by addition of a suitable acid (e.g. citric or malic acid) or base (sodium or potassium hydroxide, aminomethyl propanol)

In a suitable embodiment, a colourant is incorporated into the compositions of the invention. More suitably, the colourant is in the form of an inorganic pigment (e.g. a metal oxide) or dye that is added to the compositions to impart colour to the product and to the substrate to which it is applied (e.g. the lips).

For lip products, additional agents may be added to the formulations to increase the gloss of lips, for example, by increasing their reflectivity). These may typically find use in lip gloss lipstick or even lip balm products designed to deliver gloss to the lips and may include by way of example mother of pearl and mica or calcium sodium borosilicate glass coated with a metal oxide such as iron oxide or titanium dioxide in an appropriate particulate form, or alternatively oils and waxes or combinations thereof that accomplish this effect. Lip compositions may optionally include any agent that has a temporary or permanent lip plumping effect, for instance menthol, chilli, vanillyl butyl ether or a peptide based material such as for instance hexapeptide-3 temporary lip plumping agents may optionally work by causing irritation to the lip tissue, whereas longer lasting or more permanent effects may be observed from agents that modify the collagen or moisture composition of lips, for example. Optionally, the polymers defined herein may be mixed with the lip plumping agent before addition to the cosmetic composition to modify the agent's behaviour on the lips, for instance extending the time a temporary lip plumping agent will give a detectable benefit. Compositions may also contain an opacifying or pearlescent material.

A number of other components may be incorporated into cosmetic compositions to increase the range of benefits that the product is able to offer. These include emollients and moisturisers such as aloe vera, cocoa butter, squalane, PEG-7 Glyceryl Cocoate, Coenzyme Q-10, allantoin, sunscreens and other agents capable of blocking or assisting to block the harmful effect of the sun's light (including organic materials like oxybenzone, Padimate O or octinoxate or alternatively inorganic materials like zinc or titanium dioxide, or a combination of both inorganic and organic materials), and active ingredients capable of, or perceived, to benefit the health or appearance of the skin or to treat a disease. These ingredients include, but are not limited to, antioxidants and/or vitamins (e.g. vitamin E and its derivatives), hyaluronic acid, analgesics (e.g. camphor, menthol, phenol), collagen and its derivatives. Antioxidants such as vitamin E or butylhydroxytoluene (BHT) may also be added to avoid the composition being spoiled or altered by the oxidative degradation of its components.

Optionally, the compositions of the invention may also include fragrances and/or flavours (such as fruit, herb, vegetable, savoury or confectionary flavours) and/or sweeteners (for instance saccharin), to enhance the sensorial profile of the product making its application or use more pleasant and appealing to the consumer or user. For instance, the flavour or fragrance may be used to counteract and mask the intrinsic flavour or fragrance of the oils and waxes used in the composition or might be used to give the user the perception of a flavour on the lips. Vegetable, and particularly herb, extracts are frequently added. Compositions of the invention may optionally include egg white. Compositions of the invention may also include the use of a preservative to prevent the growth of bacteria and/or fungi in the compositions, for instance the family of alkyl parabens including methyl, ethyl, propyl and butyl paraben, diazolidinyl urea, sodium or potassium benzoate. It will be understood by those skilled in the art that some ingredients that may be added to preserve or assist in the preservation of the composition have another primary or secondary role, e.g. as an emollient. Compositions of the invention may include an agent to increase the stability of the lip product structure or the compatibility of the agents therein. Compositions of the invention may also contain film forming agents for instance clays or modified clays and polymers including stearalkonium hectorite and polybutene.

In the case of alcohol and water based formulations, for example, certain foundation and lip ink formulations, the polymer is typically dissolved or dispersed in one of the phases and then mixed with the other components of the formulation. In many cases it is easier to achieve this process by raising the temperature of the mixture, for instance to 80° C. in the case of water. It may also be advantageous to add an emulsifier to ensure better compatibility with the mixture. In the case of more hydrophobic copolymers it is often advantageous to dissolve or disperse them in the ethanol phase first if present. Water based foundation formulations may also include clays, the polymer may optionally be premixed with the clays first to increase their performance. An additional polymer system may be added to water based formulations (for instance lip make-up or foundation) to increase further their transfer resistance. Formulations may optionally include various natural or synthetic viscosity modifiers or thickeners for instance polymers like poly(acrylic acid) (marketed as carbomer), cellulosic materials like hydroxyethylcellulose, natural gums like xanthan and guar gum or clays or clay derivatives such as stearalkonium hectorite.

In a particularly suitable embodiment of the invention, the cosmetic composition is in the form of an emulsion, preferably, a water-in-oil or oil-in-water emulsion.

In an embodiment, the cosmetic or personal care composition has a pH of 1-12. Low pH compositions include skin peels, whereas high pH compositions include hair dyes and bleaches. Suitably, the cosmetic or personal care composition has a pH of 4-8. Owing to the pH of skin (which is slightly acidic), the cosmetic or personal care composition most suitably has a pH of 5-7.

In another particularly suitable embodiment, the cosmetic or personal care composition is for application to the skin, hair or nails. More suitably, the cosmetic or personal care composition is selected from the group consisting of shampoo, conditioner, hair styling products, hair spray, hair mousse, hair gel, hair wax, hair pomade, hair clay, hair volumiser, putty, a leave-on skin product (including emollients), a rinse-off skin product (including shower gels), lipstick, lip gloss, lip liner, lip plumper, lip balm, lip sheer, lip ink, lip conditioner, lip primer, lip booster, foundation, face powder, concealer, blusher, bronzer, eye shadow, eyeliner, mascara, skin peel, hair dyes and bleaches.

It will be understood that the cosmetic or personal care product does not encompass oral care products (i.e. products designed for use in or around the oral cavity). Oral care products will be understood as being distinct from make-up products that are applied to the lips (e.g. lip stick and lip balm).

In another embodiment, the cosmetic or personal care product does not include fluoride ions.

In another embodiment, the cosmetic or personal care product does not include stannous and/or zinc ions.

The quantity of film forming polymer present within the cosmetic or personal care composition varies depending on the nature of the product. In an embodiment, the cosmetic or personal care composition comprises 2-10 wt. % of a polymer defined herein. Suitably, the cosmetic or personal care composition comprises 3-8 wt. % of a polymer defined herein. More suitably, the cosmetic or personal care composition comprises 3.5-6.5 wt. % of a polymer defined herein.

In one embodiment, the cosmetic or personal care composition is a hard lip product (such as lipstick) comprising 1 to 20 wt. % of a polymer defined herein. Suitably, the cosmetic or personal care composition is a hard lip product (such as lipstick) comprising 2.5 to 15 wt. % of a polymer defined herein. More suitably, the cosmetic or personal care composition is a hard lip product (such as lipstick) comprising 2.5 to 12 wt. % of a polymer defined herein.

In one embodiment, the cosmetic or personal care composition is a soft lip product (such as lip balm) comprising 1 to 50 wt. % of a polymer defined herein. Suitably, the cosmetic or personal care composition is a soft lip product (such as lip balm) comprising 5 to 40 wt. % of a polymer defined herein. More suitably, the cosmetic or personal care composition is a soft lip product (such as lip balm) comprising 5 to 35 wt. % of a polymer defined herein. Due to the lower hardness of soft lip balm products it can be advantageous to take advantage of the ability to incorporate more polymer into the formulation. In some cases however it will be preferable to use a lower amount of polymer to allow the incorporation of more oil.

In a particularly suitable embodiment, the cosmetic or personal care compositions is a lipstick, lip gloss or lip balm base comprising:
a. a polymer as defined herein (e.g. poly(sodium itaconate) or poly(potassium itaconate)) in an amount of from 1% to 40% by weight;
b. one or more waxes, oils and fats in an amount from 10% to 50% by weight. Suitably these are typically from natural, mineral or synthetic origin such as ozokerite wax, microcrystalline wax, beeswax, carnauba wax, or candelila wax; lanolin, shea butter, castor oil, olive oil, jojoba oil, coconut oil, sesame oil, safflower oil, orange oil, mineral oil, canola oil, a silicone/methicone oil, or paraffin oil; and optionally
c. one or more ingredients with some form of antioxidant, and/or biocidal/preservative action, for instance vitamin E, vitamin E acetate, Vitamin C, BHT, an alkyl paraben such as propylparaben, methyl paraben or a mixture of parabens, diazolidinyl urea, sodium and/or potassium benzoate in an amount from 0.1% to 1% by weight.
   Optionally, the lipstick, lip gloss lip balm further comprises one or more additional suitable excipients, such as pigments, flavours, fragrances, sweeteners, UV actives and/or gloss enhancing agents.

Face cosmetics comprising polymers defined herein can be formulated into a variety of formats including liquids, powders, creams, sticks or gels according to that desired. Many of the previously discussed cosmetic ingredients used in lip products including oils waxes, pigments and fragrances may be used.

Foundation formulations may contain a range of different components depending on the physical format required of the final product. In the case of a fluid any of the oils or emolients discussed previously. In the case of a powder formulation, the foundation will be typically based on talc, optionally mixed with a range of powdered ingredients like kaolin, precipitated chalk, titanium dioxide, zinc oxide, zinc stearate, bismuth oxychloride, magnesium carbonate and magnesium stearate. In addition to acting as fillers, many of these powdered materials have secondary benefits such as preventing caking in the product (magnesium and zinc stearate) or opacifiers (magnesium carbonate). It is preferred that the polymer be premixed with the talc or powdered materials, either by adding as a melt and grinding the resulting material to the desired size or by combining the materials in a volatile medium and coating them with a suitable process such as spray drying. If it is desired that the resulting product be marketed as a mineral foundation, an inorganic material like zinc oxide, titanium dioxide or bismuth oxychloride will typically be added.

In the case of a concealer, a liquid format is typically preferred. A mixture of the oils and waxes mentioned before will typically be used. The polymers defined herein may be typically mixed into the waxes and oils as with foundation. The composition will typically be formulated with a greater level of pigment than is typical for other cosmetic products to enable it to give coverage over blemishes. This may be achieved by using comparatively large amounts of titanium dioxide, typically 10 to 30 wt. % of the composition.

Rouge or blusher may come in a range of formats including powder, cream or fluid. The polymers defined herein may typically be mixed in a similar method to that in foundation. Frequently a red pigment (e.g. iron oxide, preferably 0 to 15%) will be incorporated to give a hint of red colour to the cheeks and face.

In one embodiment, the cosmetic or personal care composition is a face product comprising 1 to 20 wt. % of a polymer defined herein. Suitably, the cosmetic or personal care composition is a face product comprising 1 to 15 wt. % of a polymer defined herein. More suitably, the cosmetic or personal care composition is a face product comprising 2 to 12 wt. % of a polymer defined herein.

Eye cosmetics comprising polymers defined herein are typically formulated into liquids, powders, or gel formats according to that desired. Many of the previously discussed cosmetic ingredients used in lip products including oils waxes, pigments and fragrances may be used. One exception is that the purity and safety of all components, particularly pigments, is of great importance.

Mascara may generally be divided into water based and non-water based transfer resistant formulations. The water based formulations will typically include surfactants to help solubilise the hydrophobic components of the system. Transfer resistant formulations will contain a volatile oil that will evaporate comparatively quickly to result in a transfer resistant film. Optionally, a mascara formulation may contain a fibre like nylon or rayon to increase the length of the lashes. Suitably, the polymers defined herein may be premixed with the fibre to increase its adhesion.

Eye shadow compositions are suitably formulated by dispersing intensely coloured inorganic pigments into a liquid or cream base in a similar manner to that used with liquid foundation. In another suitable approach, polymers defined herein are mixed in with solid ingredients like talc to make a powder formulation, using the methods discussed for making powder foundation.

Eyeliner may be dispensed from a solid pencil, made from a material like wood as an outer surface for example. The pencils containing polymers as defined herein and other ingredients may optionally be formed by mixing the materials in together in a granulated form and compressing them together. Suitably, the materials will be mixed by melting them together and intimately mixing them together. The components may also be combined by a method in which the components are continuously passed into an extruder and fed out of the other side.

Nail lacquers can be formulated by dissolving or dispersing a film former in a volatile medium. In one embodiment the solvent is comprised of a mixture of organic oils such as ethyl, propyl, butyl acetate and ethyl, propyl, n and tert butyl alcohols, in which case the polymers defined herein are predominantly hydrophobic in character. In a suitable embodiment the solvent is water and the polymer defined herein is predominantly hydrophilic in nature. One or more plasticisers are typically added to the product to provide the product with enhanced flexibility on the nail. One or more pigments may be added to the nail product in order to create a nail polish.

It will be understood that the cosmetic or personal care composition may optionally include any of the other types of cosmetically acceptable materials listed in this specification, including but not limited to waxes, oils, emulsifiers, an agent to control adhesion, agents to give skin protection from ultraviolet radiation, antioxidants, preservatives, fragrances, and pigments.

Other compositions that may typically be adapted to use the polymers defined herein may be found in “A Formulary of Cosmetic Preparations Volume. One: Decorative Cosmetics, ed. Anthony L. L. Hunting, 2^{nd} edition 2003, Micelle Press, Weymouth, England.

### Method of Preparing Cosmetic or Personal Care Compositions

As described hereinbefore, the present invention also provides a process for the preparation of a cosmetic or personal care composition, the process comprising the step of mixing a polymer as defined herein with one or more cosmetically acceptable diluents, excipients or carriers.

The cosmetic compositions of the present invention are typically prepared by melting the polymers defined herein with one or more oils and/or waxes, resulting in a homogenous product.

Suitably, lip products are formulated with a mixture of waxes, oils and fats. Many of these oils will remain on the lips during normal use. Some fatty ingredients like shea butter or lanolin may possess emollient properties as well. Optionally volatile oils (frequently based on dimethicone. for instance) may be used instead of, or in conjunction with, other oils. These oils evaporate on the surface of the lips, increasing the transfer resistance of the lip product, most typically lipstick. Some of these materials are multifunctional and also impart other benefits. For example, lanolin, petrolatum, dimethicone are recognized for their benefits in skin protection. In general the materials are adjusted to build the required physical properties of the lip product. This is typically achieved by varying the proportion of hard (higher melting/drip point) and soft (lower melting point) waxes and oils. For example, in a product like lipstick, a harder physical form is needed and a higher ratio of wax to oil is used. In a more fluid product like lip gloss, a higher proportion of oils or soft waxes is used.

In another embodiment, the process may further include the step of extruding or coextruding the mixed ingredients. Lip liner may be dispensed from a solid pencil, made from a material like wood as an outer surface for example. The polymers defined herein and other ingredients may optionally be formed by mixing the materials in together in a granulated form and compressing them together. Preferentially the materials will be mixed by melting them together and intimately mixing them together. The components may also be combined by a method in which the components are continuously passed into an extruder and fed out of the other side.

In another embodiment, the polymers defined herein may be mixed with one or more cosmetically acceptable diluents, excipients or carriers (e.g. fats, oils and waxes) at an elevated temperature. Lip products like lipstick are typically manufactured by combining the relevant oils, fats and waxes together at elevated temperature and subsequently mixing with a suitable stirrer when the waxes are sufficiently soft. In the instance of an oil or wax based product the physical properties of the polymer defined herein is preferably such that it can be added as a melt to the product, or dissolved in the molten mixture of oil and wax. The polymer may be added to the mixture in a similar manner. To aid the dispersion of the material, it is typically used in either pellet or granulated or powdered form. In some cases it can be advantageous to elevate the temperature prior to addition to the mixture to make homogenisation easier. The polymer can also be combined with a suitable oil (e.g. castor oil) or wax (e.g. beeswax) to make it easier to manipulate. For example, the product with an oil may be a paste, whereas with a wax, the product may be a faster melting solid. The temperature and mixer are chosen such that an essentially homogenous mixture of the ingredients can be efficiently formed without wasting energy or substantially degrading the components. Optionally, a specially designed lipstick kettle may be used. Suitably, the temperature is in the region of from 50° C. to 120° C., more suitably 70 to 90° C. This temperature may be maintained whilst the other ingredients (pigment, actives etc) are added and mixed in. If necessary, the temperature may be reduced to avoid excess evaporation or degradation of the fragrances or active ingredients added. Alternatively, it is possible to premix fragrances or active ingredients with the structural components before heating the mixture. For example, it is possible to premix the polymers defined herein with a fragrance, flavour or active ingredient to ensure good distribution of the agent within the final product as well as longer lasting retention of the component on the lips.

In another embodiment, the process further includes the step of cooling the molten mixture in a mould. In many cases the product is preferably poured in molten form into the final package that is used to market the product to consumers. In the case of lipstick, the mixture is preferably poured into a specially designed mould. These are typically constructed from a thermally robust material such as metal and many designs are available commercially with varying capacities. The mould is typically designed to impart a cylindrical shape to the final lipstick. The mould can be designed to shape the tip of the lipstick as desired, with different designs being familiar to those skilled in the art. The lipstick mould is frequently heated to match the temperature of the mixture being poured in to avoid too rapid solidification and defects in the resulting stick. After addition of the hot mixture, it is then allowed to cool, resulting in solidification of the mixture. Optionally, the mould and mixture may be cooled to sub ambient temperatures, thereby increasing the speed at which it sets. The lipstick is then removed from the mould and placed inside the tube in which it is to be marketed.

In an embodiment, the polymer defined herein is solubilised in water before being mixed with the other components of the cosmetic or personal care composition. Suitably, the polymer is solubilised to provide a solution containing 20-55 wt. % of the polymer in water. More suitably, the polymer is solubilised to provide a solution containing 20-30 wt. % or 45-55 wt. % of the polymer in water.

The following numbered paragraphs describe particular embodiments of the invention:
1. A cosmetic or personal care composition comprising a polymer, the polymer comprising:
   a) 10-100% of monomeric moieties formed from the polymerisation of itaconic acid salts;
   b) 0-90% of monomeric moieties formed from the polymerisation of itaconic acid, esters of itaconic acid, amides of itaconic acid and/or itaconic anhydride,
   wherein the quantity of the polymer present within the cosmetic or personal care composition is sufficient to form a film on an external surface of the human body to which the composition is applied.
2. A film forming cosmetic or personal care composition comprising a polymer, the polymer comprising:
   a) 10-100% of monomeric moieties formed from the polymerisation of itaconic acid salts;
   b) 0-90% of monomeric moieties formed from the polymerisation of itaconic acid, esters of itaconic acid, amides of itaconic acid and/or itaconic anhydride;
   wherein the quantity of the polymer present within the cosmetic or personal care composition is sufficient to form a film on an external surface of the human body to which the composition is applied.
3. The cosmetic or personal care composition as described in paragraph 1 or 2, wherein the itaconic acid salts are group 1-12 metal salts or quaternary ammonium salts.
4. The cosmetic or personal care composition as described in paragraph 1, 2 or 3, wherein the monomeric moieties formed from the polymerisation of itaconic acid salts each independently have a structure according to formula (I) shown below:

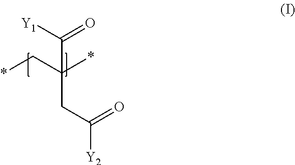

wherein
Y₁ is selected from:
   (i) a group O⁻X₁, wherein X₁ is a monovalent or divalent positively charged atom or group,
   (ii) a group O—R₁, and
   (iii) a group —N(Rₐ)(R_{b}),
      wherein R₁, Rₐ and R_{b} are each independently selected from hydrogen and (1-6C)alkyl;
Y₂ is selected from:
   (i) a group O⁻X₂, wherein X₂ is a monovalent or divalent positively charged atom or group,
   (ii) a group O—R₂, and
   (iii) a group —N(R_{c})(R_{d}),
      wherein R₂, R_{c} and R_{d} are each independently selected from hydrogen and (1-6C)alkyl,
and the monomeric moieties formed from the polymerisation of itaconic acid, esters of itaconic acid and/or amides of itaconic acid each independently have a structure according to formula (II) shown below:

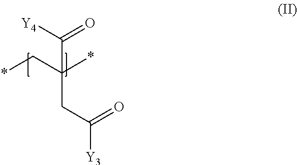

wherein
Y₃ is selected from:
   (i) a group O—R₃, and
   (ii) a group —N(Rₑ)(R_{f}),
      wherein R₃, Rₑ and R_{f} are each independently selected from hydrogen and (1-6C)alkyl; and
Y₄ is selected from:
   (i) a group O—R₄, and
   (ii) a group —N(R_{g})(Rₕ),
      wherein R₄, R_{g} and Rₕ are each independently selected from hydrogen and (1-6C)alkyl
with the proviso that at least one or Y₁ and Y₂ is a group O⁻X₁ or O⁻X₂.
5. The cosmetic or personal care composition as described in paragraph 4, wherein X₁ and X₂ are each independently selected from group 1-12 atoms and quaternary ammonium ions.
6. The cosmetic or personal care composition as described in paragraph 5, wherein the group 1-12 atoms are selected from Na, K, Mg, Ca and Zn.
7. The cosmetic or personal care composition as described in paragraph 5 or 6, wherein the group 1-12 atoms are selected from Na or K.
8. The cosmetic or personal care composition as described in paragraph 5, 6 or 7, wherein the group 1-12 atoms are Na.
9. The cosmetic or personal care composition as described in any of paragraphs 4 to 8, wherein R₁, R₂, R₃ and R₄ are each independently selected from hydrogen and (1-4C)alkyl.
10. The cosmetic or personal care composition as described in any of paragraphs 4 to 9, wherein Rₐ, R_{b}, R_{c}, R_{d}, Rₑ, R_{f}, R_{g} and Rₕ are each independently selected from hydrogen and (1-4C)alkyl.
11. The cosmetic or personal care composition as described in any of paragraphs 4 to 10, wherein Y₁ is a group O⁻X₁ and Y₂ is a group O—R₂, wherein R₂ is hydrogen.
12. The cosmetic or personal care composition as described in any of paragraphs 4 to 11, wherein Y₂ is a group O⁻X₂ and Y₁ is a group O—R₁, wherein R₁ is hydrogen.
13. The cosmetic or personal care composition as described in any of paragraphs 4 to 12, wherein the monomeric moieties formed from the polymerisation of itaconic acid salts each independently have a structure according to formula (I) shown below:

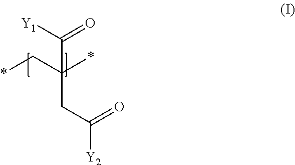

wherein
Y₁ is selected from:
   (i) a group O⁻X₁,
   (ii) a group O—R₁, wherein R₁ is hydrogen;
Y₂ is selected from:
   (i) a group O⁻X₂,
   (ii) a group O—R₂, wherein R₂ is hydrogen,
and the monomeric moieties formed from the polymerisation of itaconic acid and/or esters of itaconic acid each independently have a structure according to formula (II) shown below:

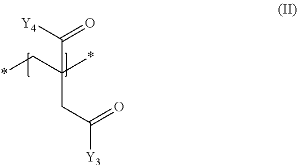

wherein
   Y₃ is a group O—R₃, and
   Y₄ is a group O—R₄;
   with the proviso that at least one or Y₁ and Y₂ is a group O⁻X₁ or O⁻X₂.
14. The cosmetic or personal care composition as described in any of paragraphs 4 to 13, wherein Y₁ is a group O⁻X₁ and Y₂ is a group O⁻X₂.
15. The cosmetic or personal care composition as described in any preceding paragraph, wherein the polymer consists essentially of monomeric moieties formed from the polymerisation of one or more of itaconic acid, sodium itaconate, potassium itaconate, ammonium itaconate, itaconic acid monomethyl ester, itaconic acid dimethyl ester, itaconic acid monoethyl ester and itaconic acid diethyl ester.
16. The cosmetic or personal care composition as described in any preceding paragraph, wherein the polymer comprises 25-100% of monomeric moieties formed from the polymerisation of itaconic acid salts.
17. The cosmetic or personal care composition as described in any preceding paragraph, wherein the polymer comprises 50-100% of monomeric moieties formed from the polymerisation of itaconic acid salts.
18. The cosmetic or personal care composition as described in any preceding paragraph, wherein the polymer comprises 90-100% of monomeric moieties formed from the polymerisation of itaconic acid salts.
19. The cosmetic or personal care composition as described in any preceding paragraph, wherein the polymer is:
   a) a homopolymer formed from the polymerisation of sodium itaconate, or
   b) a copolymer formed from the polymerisation of sodium itaconate and itaconic acid.
20. The cosmetic or personal care composition as described in any preceding paragraph, wherein the polymer has a degree of neutralisation of 50-100%.
21. The cosmetic or personal care composition as described in any preceding paragraph, wherein the polymer has a degree of neutralisation of 60-70%.
22. The cosmetic or personal care composition as described in any preceding paragraph, wherein the polymer has a molecular weight of 500-50000 Da.
23. The cosmetic or personal care composition as described in any preceding paragraph, wherein the polymer has a molecular weight of 5000-20000 Da.
24. The cosmetic or personal care composition as described in any preceding paragraph, wherein the polymer has a molecular weight of 8000-15000 Da.
25. The cosmetic or personal care composition as described in any preceding paragraph, wherein the cosmetic or personal care composition is for application to the skin, hair or nails.
26. The cosmetic or personal care composition as described in any preceding paragraph, wherein the composition has a pH of 4-8.
27. The cosmetic or personal care composition as described in any preceding paragraph, wherein the composition has a pH of 5-7.
28. The cosmetic or personal care composition of any preceding paragraph, wherein the composition comprises 1-40 wt. % of the polymer.
29. The cosmetic or personal care composition of any preceding paragraph, wherein the composition comprises 1-20 wt. % of the polymer.
30. A process for the preparation of a cosmetic or personal care composition as described in any preceding paragraph, the process comprising the steps of:
   a) providing a monomer mixture comprising water and either:
      i. one or more salts of itaconic acid,
      ii. a base, to which itaconic acid is subsequently added, or
      iii. itaconic acid, to which a base is subsequently added;
   b) reacting the mixture of step a) with a polymerisation initiator;
   c) isolating the polymeric product resulting from step b); and
   d) mixing the isolated polymeric product of step c) with one or more cosmetically acceptable diluents, excipients or carriers.
31. The process of paragraph 30, wherein step b) comprises reacting the mixture of step a) with a polymerisation initiator at a temperature of 50-100° C.
32. The process of paragraph 30 or 31, wherein step b) comprises reacting the mixture of step a) with a polymerisation initiator for a period of 2 to 96 hours.
33. The process of paragraph 30, 31 or 32, wherein the quantity of initiator used in step b) is 1-50 w/w % with respect to the total quantity of monomers present in step a).
34. A method for forming a film on an external surface of the human body, the method comprising the step of:
   a) applying to the external surface, a polymer as defined in any of paragraphs 1-29 or
      a cosmetic or personal care composition as defined in any of paragraphs 1-29, wherein the amount of polymer or personal care composition applied to the external surface is sufficient to form a film on the external surface.
35. The method of paragraph 34, wherein the external surface is selected from one or more of the skin, hair and nails.

### EXAMPLES

Examples of the invention are now provided, for the purpose of reference and illustration only.

All reagents were obtained from obtained from commercial suppliers and used without further purification.

### Synthesis of Sodium Itaconate

Itaconic acid (200.00 g, 1.537 mol) and deionised water (800.00 g) were charged to a 2 litre glass beaker. 47% sodium hydroxide solution (177.66 g, 2.088 mol of NaOH) was added slowly with stirring from a large magnetic flea. The final pH of the solution was 6.0. The solution was dried on a rotary evaporator at 60° C. and then dried in a vacuum oven at 40° C. for 24 hours. Product yield was 248.50 g.

Analysis by FTIR showed a strong C═O carboxylate peak at 1558.9 cm⁻¹ and reduction of the C═O carboxylic acid peak at 1685.8 cm⁻¹.

### Synthesis of Potassium Itaconate

Itaconic acid (50.00 g, 0.382 moles) and deionised water (450.00 g) were charged to a 1 litre glass beaker. 85% potassium hydroxide (33.55 g, 0.508 moles KOH) was added slowly with stirring from a large magnetic flea. The final pH of the solution was 5.5-6.0.

The solution was dried on a rotary evaporator at 60° C. and then dried in a vacuum oven at 40° C. for 24 hours. Product yield was 78.32 g.

Analysis by FTIR showed a strong C═O carboxylate peak at 1551.5 cm⁻¹ and reduction of the C═O carboxylic acid peak at 1685.8 cm⁻¹.

### Synthesis of Monethyl Itaconate

Ethanol, 200 proof (450.00 g, 9.768 mol) was charged to a 1 L flange flask fitted with an overhead stirrer, a condenser and a steady nitrogen flow. Itaconic anhydride (50.00 g, 0.446 mol) was added to the flask. The flask was heated by an oil bath to an external temperature of 95° C. Temperature was maintained for 16 hours.

The solution was allowed to cool to ambient temperature. Ethanol was removed by rotary evaporation at 40° C. and the product was dried in a vacuum oven at 40° C. for 24 hours. Product yield was 60.02 g.

Analysis by FTIR showed a strong C═O ester peak at 1723.6 cm⁻¹ and a C═O carboxylic acid peak at 1692.2 cm⁻¹ and reduction of the C═O anhydride peaks at 1842.9 cm⁻¹ and 1762.8 cm⁻¹.

### Polymerisation Processes

### Example 1

### Polymerisation of Sodium Itaconate with Itaconic Acid

Deionised water (310.00 g) was charged to a 1 litre flange flask fitted with an overhead stirrer, condenser and steady nitrogen flow. The deionised water was flushed with nitrogen for 5 minutes. Itaconic acid (93.00 g, 0.715 mol) was added to the flask. The external temperature of the flask was increased to 60° C. using a heated oil bath. Once the itaconic acid had dissolved, initial solution pH was 1. 47% sodium hydroxide (9.91 g, 0.116 mol) was slowly added to the flask to adjust the pH to 2. Potassium persulfate (3.68 g, 0.0136 mol) was then added and the external temperature was increased to 65° C. Temperature was maintained for 48 hours. The solution was allowed to cool to ambient temperature. The polymer was then precipitated in acetone (1 litre). The precipitate was washed with acetone (2×100 mL) and dried in a vacuum oven at 40° C. for 24 hours. Polymer yield was 82.95 g.

### Example 2

### Synthesis of Poly(Sodium Itaconate) Using Sodium Persulfate Initiator

Deionised water (233.85 g) was charged to a 2 litre water jacketed glass reactor fitted with a condenser, steady nitrogen flow and overhead stirrer (210 rpm). 50% sodium hydroxide (530.10 g, 6.627 moles) was slowly added to the reactor over a period of 30 minutes. Itaconic acid (634.95 g, 4.881 moles) was then added over a period of 30 minutes. The temperature of the water jacket was increased to 73° C. to give an internal temperature of approximately 70° C.

Sodium persulfate (47.90 g, 0.201 moles) was dissolved in deionised water (102.10 g). A syringe pump was used to feed this solution into the reactor over 2 hours. Once addition of the sodium persulfate solution had been completed, temperature was maintained for an additional 4 hours. The solution product was diluted with deionised water (500.00 g). Once the solution was homogenous, it was discharged from the reactor.

Analysis of the product by HPLC determined that 93.8% of monomer had been converted to polymer.

### Example 3

### Synthesis of Poly(Sodium Itaconate) Using Sodium Persulfate Initiator at 60% Solids

Deionised water (15.38 g) and 50% sodium hydroxide solution (106.20 g, 1.328 mol) were charged to a 700 mL flange flask fitted with an overhead stirrer, condenser and steady nitrogen flow. Itaconic acid (126.99 g, 0.976 mol) was added to the flask. The internal temperature was increased to 70° C. Sodium persulfate (9.58 g, 0.040 mol) was dissolved in deionised water (20.42 g). A syringe pump was used to feed this solution into the flask over 2 hours. Once addition of the sodium persulfate solution had been completed, temperature was maintained for an additional 4 hours. The solution was discharged from the flask and allowed to cool to ambient temperature.

Analysis of the product by HPLC determined that 99.8% of monomer had been converted to polymer.

### Example 4

### Synthesis of Poly(Sodium Itaconate) Using Sodium Persulfate Initiator at 57% Solids

Deionised water (50.50 g) and 50% sodium hydroxide solution (159.30 g, 1.992 mol) were charged to a 700 mL flange flask fitted with an overhead stirrer, condenser and steady nitrogen flow. Itaconic acid (190.49 g, 1.464 mol) was added to the flask. The internal temperature was increased to 70° C. Sodium persulfate (14.37 g, 0.060 mol) was dissolved in deionised water (25.25 g). A syringe pump was used to feed this solution into the flask over 2 hours. Once addition of the sodium persulfate solution had been completed, temperature was maintained for an additional 4 hours. The solution was discharged from the flask and allowed to cool to ambient temperature.

Analysis of the product by HPLC determined that 97.8% of monomer had been converted to polymer.

### Example 5

### Synthesis of Poly(Sodium Itaconate) Using Sodium Persulfate Initiator at 45% Solids

Deionised water (510.63 g) was charged to a 2 litre water jacketed glass reactor fitted with a condenser, steady nitrogen flow and overhead stirrer (210 rpm). 50% sodium hydroxide (530.10 g, 6.627 mol) was added slowly to the reactor over 25 minutes. Itaconic acid (634.95 g, 4.881 mol) was added over 45 minutes. After addition was completed, the internal temperature was adjusted to 70° C. Sodium persulfate (47.90 g, 0.201 mol) was dissolved in DI water (102.10 g). A syringe pump was used to feed this solution into the reactor over 2 hours. After a further 4 hours at temperature the solution was allowed to cool to ambient temperature with overhead stirring before being discharged from the reactor.

Analysis of the product by HPLC determined that 94.7% of monomer had been converted to polymer.

### Example 6

### Synthesis of Poly(Sodium Itaconate) Using Sodium Persulfate Initiator, 8 Hour Reaction

Deionised water (232.85 g) was charged to a 2 litre water jacketed glass reactor fitted with a condenser, steady nitrogen flow and overhead stirrer (200 rpm). 50% sodium hydroxide solution (530.10 g, 6.627 mol) and itaconic acid (634.95 g, 4.881 mol) were added to the flask over 45 minutes. The monomer solution was stirred in the reactor at ambient temperature overnight.

The following morning, the internal temperature was increased to 70° C. Sodium persulfate (47.90 g, 0.201 mol) was dissolved in DI water (102.10 g). A syringe pump was used to feed this solution into the reactor over 2 hours.

After a further 4 hours at temperature the reaction solution was diluted with DI water (500.00 g). The internal temperature was reduced to 60° C. Once the solution was homogeneous an aliquot was taken. After 2 hours at 60° C. the solution was discharged from the reactor whilst hot.

Analysis of the product by HPLC determined that 98.7% of monomer had been converted to polymer.

### Example 7

### Synthesis of Poly(Sodium Itaconate) at Reflux with Staggered Initiator Addition

Deionised water (46.57 g) and 50% sodium hydroxide solution (106.20 g, 1.328 mol) were charged to a 500 mL flange flask fitted with an overhead stirrer, condenser and steady nitrogen flow. Itaconic acid (126.99 g, 0.976 mol) was added to the flask. The internal temperature was increased to 98° C. Sodium persulfate (9.58 g, 0.040 mol) was dissolved in deionised water (20.42 g). A syringe pump was used to feed this solution into the reactor over 2 hours. Temperature of the flask was maintained for a further 30 minutes. The solution was then allowed to cool to ambient temperature.

Analysis of the product by HPLC determined that 95.8% of monomer had been converted to polymer.

### Example 8

### Synthesis of Poly(Sodium Itaconate) at Reflux with Staggered Initiator Addition and Monomer Burn-Out

Deionised water (46.57 g) and 50% sodium hydroxide solution (106.20 g, 1.328 mol) were charged to a 500 mL flange flask fitted with an overhead stirrer, condenser and steady nitrogen flow. Itaconic acid (126.99 g, 0.976 mol) was added to the flask. The internal temperature was increased to 98° C. Sodium persulfate (9.58 g, 0.040 mol) was dissolved in deionised water (20.42 g). A syringe pump was used to feed this solution into the reactor over 2 hours. Temperature of the flask was maintained for a further 30 minutes. An aliquot of solution was taken. Sodium persulfate (1.65 g, 6.93 mmol) was added to the flask. Temperature was maintained for one hour. The solution was allowed to cool to ambient temperature.

Analysis of the product by HPLC determined that 96.8% of monomer had been converted to polymer.

### Example 9

### Synthesis of Poly(Sodium Itaconate), 52.5% Solids, 6.6% Initiator, 70° C.

Deionised water (33.50 g) and 50% sodium hydroxide solution (53.01 g, 0.664 mol) were charged to a 250 mL flange flask fitted with a condenser, steady nitrogen flow and overhead stirrer.

Itaconic acid (63.50 g, 0.488 mol) was added to the flask. The internal temperature of the flask was increased to 70° C. Sodium persulfate (5.94 g, 0.0249 mol) was added to the flask. After 6 hours at temperature the solution was diluted with deionised water (46.87 g). The internal temperature was then increased to 95° C. This temperature was maintained for one hour. The solution was then allowed to cool to ambient temperature.

Analysis of the product by HPLC determined that 99.0% of monomer had been converted to polymer.

### Example 10

### Synthesis of Poly(Sodium Itaconate), Initiator Solution Added Over 3 Hours

Deionised water (46.57 g) and 50% sodium hydroxide solution (106.20 g, 1.328 mol) were charged to a 700 mL flange flask fitted with an overhead stirrer, condenser and steady nitrogen flow. Itaconic acid (126.99 g, 0.976 mol) was added to the flask. The internal temperature was increased to 70° C. Sodium persulfate (9.58 g, 0.040 mol) was dissolved in deionised water (20.42 g). A syringe pump was used to feed this solution into the flask over 3 hours. Once addition of the sodium persulfate solution had been completed, temperature was maintained for an additional 3 hours. The solution was discharged from the flask and allowed to cool to ambient temperature.

Analysis of the product by HPLC determined that 98.2% of monomer had been converted to polymer.

### Example 11

### Synthesis of Poly(Sodium Itaconate), Initiator Solution Added Over 1 Hour

Deionised water (46.57 g) and 50% sodium hydroxide solution (106.20 g, 1.328 mol) were charged to a 700 mL flange flask fitted with an overhead stirrer, condenser and steady nitrogen flow. Itaconic acid (126.99 g, 0.976 mol) was added to the flask. The internal temperature was increased to 70° C. Sodium persulfate (9.58 g, 0.040 mol) was dissolved in deionised water (20.42 g). A syringe pump was used to feed this solution into the flask over 1 hour. Once addition of the sodium persulfate solution had been completed, temperature was maintained for an additional 5 hours. The solution was discharged from the flask and allowed to cool to ambient temperature.

Analysis of the product by HPLC determined that 97.7% of monomer had been converted to polymer.

### Example 12

### Synthesis of Poly(Sodium Itaconate) Using a Reflux, 5.8% Sodium Persulfate, 28.8% Solids

Deionised water (37.50 g) was charged to a 250 mL flange flask fitted with a condenser, steady nitrogen flow and overhead stirrer. Itaconic acid (66.25 g, 0.509 mol), deionised water (40.00 g) and 50% sodium hydroxide solution (40.00 g, 0.500 mol) were added to the flask.

An initiator solution of sodium persulfate (5.00 g, 0.021 mol) and deionised water (18.75 g) was prepared. The internal temperature was increased to 98° C. The initiator solution was added over a period of 2 hours. Temperature was maintained for an additional 30 minutes. The solution was then allowed to cool to ambient temperature. The solution was then diluted with deionised water (62.5 g).

Analysis of the product by HPLC determined that 86.3% of monomer had been converted to polymer.

### Example 13

### Synthesis of Poly(Sodium Itaconate) Using Potassium Persulfate Initiator

Deionised water (334.95 g) was charged to a 2 litre water jacketed glass reactor fitted with a condenser, steady nitrogen flow and overhead stirrer. 50% sodium hydroxide solution (530.10 g, 6.627 mol) was added to the reactor over a period of 30 minutes. Itaconic acid (634.95 g, 4.881 mol) was then added over a period of 30 minutes. The temperature of the water jacket was increased to 73° C. to give an internal temperature of approximately 70° C.

Potassium persulfate (67.50 g, 0.250 mol) was added to the reactor over a period of 15 minutes. Temperature of the reaction solution was maintained for 6 hours. Deionised water (500.00 g) was added to dilute the reaction solution. The solution was then discharged from the reactor

Analysis of the product by HPLC determined that 98.5% of monomer had been converted to polymer.

### Example 14

### Synthesis of Poly(Sodium Itaconate), 52.5% Solids, 70° C., 7.5% Initiator

Deionised water (66.99 g) and 50% sodium hydroxide solution (106.02 g, 1.325 mol) were charged to a 500 mL flange flask fitted with a condenser, steady nitrogen flow and overhead stirrer.

Itaconic acid (126.99 g, 0.976 mol) was added to the flask. Potassium persulfate (12.15 g, 0.045 mol) was added to the flask. The internal temperature was increased to 70° C. Temperature was maintained for six hours. The internal temperature was then increased to 95° C. Potassium persulfate (1.35 g, 0.005 mol) was added to the flask. Temperature was maintained for a further 60 minutes. The solution was allowed to cool to ambient temperature before being discharged from the flask.

Analysis of the product by HPLC determined that 98.8% of monomer had been converted to polymer.

### Example 15

### Synthesis of Poly(Sodium Itaconate), 52.5% Solids, 60° C., 6.75% Initiator

Deionised water (66.99 g) and 50% sodium hydroxide solution (106.02 g, 1.325 mol) were charged to a 500 mL flange flask fitted with a condenser, steady nitrogen flow and overhead stirrer.

Itaconic acid (126.99 g, 0.976 mol) was added to the flask. Potassium persulfate (9.72 g, 0.036 mol) was added to the flask. The internal temperature was increased to 60° C. Temperature was maintained for 21 hours. Internal temperature was then increased to 95° C. and maintained for 1 hour. The solution was allowed to cool to 72° C. and was diluted with deionised water (75.00 g) before discharging from the flask.

Analysis of the product by HPLC determined that 99.1% of monomer had been converted to polymer.

### Example 16

### Synthesis of Poly(Sodium Itaconate), 52.5% Solids, 60° C., 7.5% Initiator and Monomer Burn

Deionised water (66.99 g) and 50% sodium hydroxide solution (106.02 g, 1.325 mol) were charged to a 500 mL flange flask fitted with a condenser, steady nitrogen flow and overhead stirrer.

Itaconic acid (126.99 g, 0.976 mol) was added to the flask. Potassium persulfate (12.15 g, 0.045 mol) was added to the flask. The internal temperature was increased to 60° C. Temperature was maintained for 6 hours. Internal temperature was then increased to 95° C. and potassium persulfate (1.35 g, 4.994 mmol) was added. Temperature was maintained for one hour and was then allowed to cool to ambient temperature.

Analysis of the product by HPLC determined that 98.9% of monomer had been converted to polymer.

### Example 17

### Synthesis of Poly(Sodium Itaconate), 52.5% Solids, 70° C., 2% Initiator and Monomer Burn

Deionised water (120.00 g) and sodium hydroxide (53.01 g, 1.325 mol) were charged to a 500 mL flange flask fitted with a condenser, steady nitrogen flow and overhead stirrer. Itaconic acid (126.99 g, 0.976 mol) was added to the flask. Potassium persulfate (3.24 g, 0.012 mol) was added to the flask.

The internal temperature was increased to 70° C. Temperature was maintained for 6 hours. Internal temperature was then increased to 95° C. and potassium persulfate (0.36 g, 1.332 mmol) was added. Temperature was maintained for one hour and was then allowed to cool to ambient temperature.

Analysis of the product by HPLC determined that 93.5% of monomer had been converted to polymer.

### Example 18

### Synthesis of Poly(Sodium Itaconate) Using Hydrogen Peroxide Initiator—10% Peroxide, 80° C.

A 2 litre water jacketed reactor was charged with itaconic acid (474.25 g, 3.65 mol), iron (III) ammonium sulfate dodecahydrate (70 mg, 0.145 mmol) and deionised water (474.25 g). The water bath was then set at 7° C. and the mixture was stirred for 5 minutes under an atmosphere of nitrogen using an overhead stirrer. To the mixture was then added 50% sodium hydroxide solution (393.61 g, 4.92 mol) dropwise. After addition of sodium hydroxide solution, the mixture was warmed to 80° C. (85° C. water bath temperature). 30% hydrogen peroxide solution (157.85 g, 1.39 mol) was then added to the mixture dropwise over a period of 45-60 minutes whilst stirring under an atmosphere of nitrogen. After the addition was complete, the reaction was heated at 80° C. under nitrogen accompanied by stirring for a further 20 hours at which point it was determined that all hydrogen peroxide had decomposed. The product was then allowed to cool to room temperature before being discharged from the reactor.

Analysis of the product by HPLC determined that 95.3% of monomer had been converted to polymer.

### Example 19

### Synthesis of Poly(Sodium Itaconate) Using Hydrogen Peroxide Initiator—29% Peroxide, Reflux

A 500 mL flange flask was charged with itaconic acid (135.50 g, 1.042 mol) and ferric ammonium sulfate dodecahydrate (0.02 g, 0.041 mmol) and deionised water (42.50 g). The flask was fitted with a condenser, overhead stirrer and steady nitrogen flow. 50% sodium hydroxide solution (33.30 g, 0.416 mol) was added dropwise. A heating block was used to increase the temperature of the flask until the mixture was refluxing (external temperature of 110° C.). 30% hydrogen peroxide solution (132.40 g, 1.168 mol) was added dropwise over 2 hours. Temperature was maintained for an additional 2 hours. The mixture was allowed to cool to ambient temperature. Analysis with peroxide dip-strips indicated that the sample contained 15 g/L hydrogen peroxide.

Half of the reaction mixture was heated for an additional 16 hours before being allowed to cool to ambient temperature, reducing hydrogen peroxide levels to <0.5 mg/L.

Analysis of the product by HPLC determined that 100.0% of monomer had been converted to polymer.

### Example 20

### Synthesis of Poly(Sodium Itaconate) Using Hydrogen Peroxide Initiator—10% Peroxide, 70° C.

A 250 mL flange flask was charged with itaconic acid (67.75 g, 0.521 mol), ferric ammonium sulfate dodecahydrate (0.01 g, 0.021 mmol) and deionised water (67.75 g). The flask was fitted with a condenser, overhead stirrer and steady nitrogen flow. 50% sodium hydroxide solution (56.23 g, 0.703 mol) was slowly added to the solution. The internal temperature of the flask was increased to 70° C. 30% hydrogen peroxide solution (22.55 g, 0.199 mol) was added dropwise over 30 minutes. Temperature was maintained for an additional 40 hours before being allowed to cool to ambient temperature. Analysis with peroxide dip strips confirmed that there was no hydrogen peroxide remaining in the sample.

Analysis of the product by HPLC determined that 98.5% of monomer had been converted to polymer.

### Example 21

### Synthesis of Poly(Sodium Itaconate) Using Hydrogen Peroxide Initiator—20% Peroxide, Reflux

A 250 mL flange flask was charged with itaconic acid (67.75 g, 0.521 mol), ferric ammonium sulfate dodecahydrate (0.01 g, 0.021 mmol) and deionised water (67.75 g). The flask was fitted with a condenser, overhead stirrer and steady nitrogen flow. 50% sodium hydroxide solution (56.23 g, 0.703 mol) was slowly added to the solution. The internal temperature of the flask was increased to reflux. 30% hydrogen peroxide solution (45.10 g, 0.398 mol) was added dropwise over 60 minutes. Temperature was maintained for 16 hours before being allowed to cool to ambient temperature. Analysis with peroxide dip strips confirmed that there was no hydrogen peroxide remaining in the sample.

Analysis of the product by HPLC determined that 99.8% of monomer had been converted to polymer.

### Example 22

### Synthesis of Poly(Sodium Itaconate) Using Hydrogen Peroxide Initiator—20% Peroxide, 80° C.

A 250 mL flange flask was charged with itaconic acid (67.75 g, 0.521 mol), ferric ammonium sulfate dodecahydrate (0.01 g, 0.021 mmol) and deionised water (67.75 g). The flask was fitted with a condenser, overhead stirrer and steady nitrogen flow. 50% sodium hydroxide solution (56.23 g, 0.703 mol) was slowly added to the solution. The internal temperature of the flask was increased to 80° C. 30% hydrogen peroxide solution (45.10 g, 0.398 mol) was added dropwise over 90 minutes. Temperature was maintained for 30 hours before being allowed to cool to ambient temperature. Analysis with peroxide dip strips confirmed that there was no hydrogen peroxide remaining in the sample.

Analysis of the product by HPLC determined that 100.0% of monomer had been converted to polymer.

### Example 23

### Polymerization of Potassium Itaconate

Deionised water (66.60 g) was charged to a 250 mL flange flask fitted with a condenser, steady nitrogen flow and overhead stirrer. Potassium itaconate (20.00 g, 0.111 mol, 67 mol % neutralised) was added to the flask. Temperature of the flask was increased to 65° C. Potassium persulfate (0.794 g, 2.94 mmol) was added to the flask. Temperature was maintained for 48 hours. The solution was then allowed to cool to ambient temperature. The polymer was precipitated in acetone (600 mL) and then washed with acetone (2×100 mL) and dried under vacuum. Product yield was 18.80 g.

### Example 24

### Polymerization of Ammonium Itaconate

Deionised water (631.35 g) was charged to a 2 litre water jacketed glass reactor fitted with a condenser, steady nitrogen flow and overhead stirrer. Ammonium hydroxide solution (299.49 g, 28.0-30.0% NH₃ basis) was added to the reactor over a period of 30 minutes. Itaconic acid (488.91 g, 3.758 mol) was then added over a period of 30 minutes. The monomer solution was stirred in the reactor at ambient temperature overnight.

The following morning, the temperature of the water jacket was increased to 73° C. to give an internal temperature of approximately 70° C.

Ammonium persulfate (35.32 g, 0.155 mol) was dissolved in deionised water (70.64 g). A syringe pump was used to feed this ammonium persulfate solution into the reactor over 2 hours. Temperature of the flask was maintained for a further 4 hours. The solution was then discharged from the reactor whilst hot.

Analysis of the product by HPLC determined that 86.6% of monomer had been converted to polymer.

### Example 25

### Co-Polymerization of Monoethyl Itaconate and Sodium Itaconate (50:50 Mol %)

Deionised water (51.62 g) was charged to a 250 mL flange flask fitted with a condenser, steady nitrogen flow and overhead stirrer. Sodium itaconate (8.26 g, 0.050 mol-80 mol % neutralisation) and monoethyl itaconate (7.90 g, 0.050 mol) were added to the flask. Temperature of the flask was increased to 65° C. Potassium persulfate (0.615 g, 2.28 mmol) was added to the flask. Temperature was maintained for 48 hours. The solution was then allowed to cool to ambient temperature. The post-reaction solution was dried down using a rotary evaporator and the polymer was dried under vacuum. Product yield was 11.58 g.

### Example 26

### Co-Polymerization of Sodium Itaconate and Monoethyl Itaconate (80:20 Mol %)

Deionised water (70.89 g) was charged to a 250 mL flange flask fitted with a condenser, steady nitrogen flow and overhead stirrer. Sodium itaconate (13.22 g, 0.080 mol-80 mol % neutralisation) and monoethyl itaconate (3.16 g, 0.020 mol) were added to the flask. Temperature of the flask was increased to 65° C. Potassium persulfate (0.648 g, 2.40 mmol) was added to the flask. Temperature was maintained for 48 hours. The solution was then allowed to cool to ambient temperature. The polymer was precipitated in acetone (600 mL) and then washed with acetone (2×100 mL) and dried under vacuum. Yield of the polymer was 14.63 g.

### Example 27

### Copolymerization of Itaconic Acid, Sodium Itaconate and Monoethyl Itaconate (75:25 Molar Ratio)

Deionised water (91.32 g) was charged to a 250 mL flange flask fitted with a condenser, steady nitrogen flow and overhead stirrer. Itaconic acid (19.50 g, 0.150 mol) and monoethyl itaconate (7.90 g, 0.050 mol) were added to the flask. Temperature of the flask was increased to 60° C. 47% sodium hydroxide solution (4.79 g, 0.0563 mol) was added dropwise to adjust the pH from 1.0 to 3.0. Temperature of the flask was then increased to 65° C. Potassium persulfate (0.773 g, 2.86 mmol) was added to the flask. Temperature was maintained for 48 hours. The solution was then allowed to cool to ambient temperature. The polymer was precipitated in acetone (600 mL) and then washed with acetone (2×100 mL) and dried under vacuum. Product yield was 26.74 g.

### Example 28

### Copolymerization of Itaconic Acid, Sodium Itaconate and Monoethyl Itaconate (25:75 Molar Ratio)

Deionised water (69.60 g) was charged to a 250 mL flange flask fitted with a condenser, steady nitrogen flow and overhead stirrer. Itaconic acid (4.33 g, 0.033 mol) and monoethyl itaconate (15.80 g, 0.100 mol) were added to the flask. Temperature of the flask was increased to 60° C. 47% sodium hydroxide solution (1.58 g, 0.0189 mol) was added dropwise to adjust the pH from 1.0 to 3.0. Temperature of the flask was then increased to 65° C. Potassium persulfate (0.514 g, 1.90 mmol) was added to the flask. Temperature was maintained for 48 hours. The solution was then allowed to cool to ambient temperature. The post-reaction solution was dried down using a rotary evaporator and the polymer was dried under vacuum. Product yield was 19.80 g.

### Cosmetic Compositions

The cosmetic compositions of the invention are illustrated using the following non-limiting examples. In some instances a solution of the itaconate salt polymer was used. These may be made by dissolving an appropriate amount of polymer.

### Example 29

### Black Mascara Formulation

| | Example 29 | CONTROL |
|---|---|---|
| Deionised water | 61.0% | 48.0% |
| TEGO Carbomer 141 | 2.0% | 0.0% |
| Carbopol Ultrez 10 solution | 0.0% | 25.0% |
| Example 2 - 25 weight % | 20.0% | 0.0% |
| solution in water | | |
| PVP/VA, 50% (W-735) | 0.0% | 10.0% |
| Organic glycerin | 0.5% | 0.5% |
| Tween 20 | 0.5% | 0.5% |
| Euxyl K712 | 1.0% | 1.0% |
| Mica Black | 15.0% | 15.0% |

The solution of poly(sodium itaconate) (Example 2) was diluted with all of the additional deionised water. TEGO Carbomer 141 was added slowly and the mixture was shear stirred at 5,000 rpm until homogenous using an IKA Ultra-Turrax homogeniser. Glycerin, Euxyl K712 and Tween 20 were then added and the mixture was shear stirred for one minute at 5,000 rpm. Mica black was added with manual stirring until homogenous. The resulting composition with poly(sodium itaconate) gave equal or superior performance to the control on test eye lashes

### Example 30

### Skin-Tightening Serum

| | Example 30 | CONTROL |
|---|---|---|
| Deionised water | 74.35% | 94.35% |
| TEGO Carbomer 141 | 2.5% | 2.5% |
| Example 2 (25% solution) | 20.0% | 0.0% |
| Organic glycerin | 2.0% | 2.0% |
| Euxyl K712 | 1.0% | 1.0% |
| 0.1% FD&C Blue 1 | 0.15% | 0.15% |

The solution of poly(sodium itaconate) (Example 2) was diluted with all of the additional deionised water. To this TEGO Carbomer 141 was slowly added and shear stirred for a total of 15 minutes at 10,000 RPM using an IKA Ultra Turrax homegeniser. Glycerin, FD&C Blue 1 dye and Euxyl K712 were added. The mixture was shear stirred for a further 2 minutes at 10,000 rpm.

The formulation of Example 30 was tested on the skin and found to have better application and substantivity than the control without the polymer of Example 2.

### Example 31

### Liquid Eye Shadow

| | Example 31 | CONTROL |
|---|---|---|
| Deionised water | 55.0% | 75.0% |
| TEGO Carbomer 141 | 1.0% | 1.0% |
| Organic glycerin | 3.0% | 3.0% |
| Example 2 (25% solution) | 20.0% | 0.0% |
| Prestige bright bronze | 5.0% | 5.0% |
| Mirage sparkling silver | 15.0% | 15.0% |
| Euxyl K712 | 1.0% | 1.0% |

The solution of poly(sodium itaconate) (Example 2) was diluted with all of the additional deionised water. To this TEGO Carbomer 141 was slowly added and shear stirred for 5 minutes at 5,000 RPM using an IKA Ultra-Turrax homogeniser. Organic glycerin was then added and shear stirred for 2 minutes at 10,000 rpm.

The pigments were added with manual stirring from a spatula. Once homogenous, Euxyl K712 was added and the formulation was stirred for an additional 2 minutes at 5,000 rpm.

The formulation of Example 31 was tested on the skin and found to have better application and substantivity than the control without the polymer of Example 2.

### Example 32

### Hair Gel

A hair gel containing the itaconate salt polymers was prepared according to the following formulation:

Phase A

| | |
|---|---|
| 1) Deionized water | 67.50% |
| 2) TEGO Carbomer 141 | 1.20% |
| 3) Sodium hydroxide, 25 w/w % aqueous solution | 2.70% |

Phase B

| | |
|---|---|
| 1) Example 13 polymer, 50 w/w % aqueous solution | 16.00% |
| 2) Alcohol, denatured | 10.00% |
| 3) TAGAT O 2 V (PEG-20 glycerly oleate) | 2.00% |
| 4) Perfume | 0.30% |
| 5) ABIL B 88183 (PEG/PPG-20/6 dimethicone) | 0.30% |

Phases A and B were mixed separately. Phase B was added to Phase A with agitation from a laboratory homogeniser (10,000 rpm) for 10 minutes.

To prepare Phase A, TEGO Carbomer 141 was stirred into deionized water and left to swell for one hour. High shear stirring (10,000 rpm) was applied for 20 minutes to ensure all of the Carbomer had hydrolysed. NaOH was then added whilst the solution was agitated with shear stirring, causing a pH rise from 3.0 to 5.5.

For phase B, ingredients were added sequentially in the order above. After each ingredient was added, the formulation was mixed using a laboratory homogeniser (10,000 rpm) for one minute, until uniform. After the final ingredient was added, the formulation was mixed for five minutes under high shear (10,000 rpm) to ensure thorough mixing.

The hair gel was tested by application to tresses comprised of human hair and the product allowed to dry. The polymer of Example 13 was found to give the formulation good firm hold to the hair.

### Example 32

### Aqueous Hair Spray

A simple aqueous hair spray formulation was prepared to illustrate the invention. The formulation was mixed with a laboratory homogeniser (set to 10,000 rpm) to ensure thorough mixing.

| | | |
|---|---|---|
| 1. | Deionized water | 94.95% |
| 2. | Example 13 polymer | 5.00% |
| 3. | Orange flower water fragrance | 0.05% |

The hair spray was tested by application to tresses comprised of human hair styled in various positions and the product was allowed to dry. The polymer of Example 13 was found to give good style retention.

### Example 33

### Hair Mousse

A hair mouse containing the itaconate salt polymers was prepared according to the following formulation:

| | | |
|---|---|---|
| 1. | Deionised water | 82.75% |
| 2. | Example 13 polymer (26 w/w % aqueous solution) | 15.00% |
| 3. | Glycerin | 1.00% |
| 4. | Silsense Copolyol-1 Silicone | 0.50% |
| | (PEG-33 (and) PEG-8 dimethicone (and) PEG-14) | |
| 5. | Euxyl K 300 | 0.50% |
| | (Phenoxyethanol, methyl-, ethyl-, butyl-, propyl-, | |
| | (and) isobutylparaben) | |
| 6. | Promois WG | 0.25% |
| | (Hydrolyzed wheat protein) | |

Ingredients were added in the order stated above. After each ingredient was added, the formulation was mixed under high shear (10,000 rpm) for one minute, until uniform. After the final ingredient was added, the formulation was mixed for five minutes under high shear (10,000 rpm) to ensure thorough mixing.

The resulting formulation was a clear low viscosity liquid that could be used to style hair tresses.

While specific embodiments of the invention have been described herein for the purpose of reference and illustration, various modifications will be apparent to a person skilled in the art without departing from the scope of the invention as defined by the appended claims.

1. A cosmetic or personal care composition comprising a polymer, the polymer comprising:
a) 10-100% of monomeric moieties formed from the polymerisation of itaconic acid salts;
b) 0-90% of monomeric moieties formed from the polymerisation of itaconic acid, esters of itaconic acid, amides of itaconic acid and/or itaconic anhydride,
wherein the quantity of the polymer present within the cosmetic or personal care composition is sufficient to form a film on an external surface of the human body to which the composition is applied.

2. A film forming cosmetic or personal care composition comprising a polymer, the polymer comprising:
c) 10-100% of monomeric moieties formed from the polymerisation of itaconic acid salts;
d) 0-90% of monomeric moieties formed from the polymerisation of itaconic acid, esters of itaconic acid, amides of itaconic acid and/or itaconic anhydride;
wherein the quantity of the polymer present within the cosmetic or personal care composition is sufficient to form a film on an external surface of the human body to which the composition is applied.

3. The cosmetic or personal care composition as claimed in claim 1, wherein the monomeric moieties formed from the polymerisation of itaconic acid salts each independently have a structure according to formula (I) shown below: 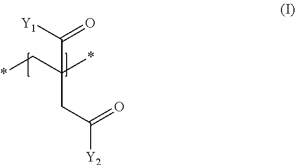
wherein
Y₁ is selected from:
(i) a group O⁻X₁, wherein X₁ is a monovalent or divalent positively charged atom or group,
(ii) a group O—R₁, and
(iii) a group —N(Rₐ)(R_{b}),
wherein R₁, Rₐ and R_{b} are each independently selected from hydrogen and (1-6C)alkyl;
Y₂ is selected from:
(i) a group O⁻X₂, wherein X₂ is a monovalent or divalent positively charged atom or group,
(ii) a group O—R₂, and
(iii) a group —N(R_{c})(R_{d}),
wherein R₂, R_{c} and R_{d} are each independently selected from hydrogen and (1-6C)alkyl,
and the monomeric moieties formed from the polymerisation of itaconic acid, esters of itaconic acid and/or amides of itaconic acid each independently have a structure according to formula (II) shown below: 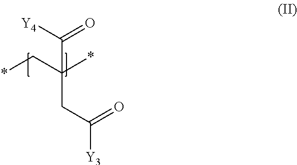
wherein
Y₃ is selected from:
(i) a group O—R₃, and
(ii) a group —N(Rₑ)(R_{f}),
wherein R₃, Rₑ and R_{f} are each independently selected from hydrogen and (1-6C)alkyl; and
Y₄ is selected from:
(i) a group O—R₄, and
(ii) a group —N(R_{g})(Rₕ),
wherein R₄, R_{g} and Rₕ are each independently selected from hydrogen and (1-6C)alkyl
with the proviso that at least one or Y₁ and Y₂ is a group O⁻X₁ or O⁻X₂.

4. The cosmetic or personal care composition as claimed in claim 3, wherein X₁ and X₂ are each independently selected from Na, K or a quaternary ammonium ion.

5. The cosmetic or personal care composition as claimed in claim 3, wherein R₁, R₂, R₃ and R₄ are each independently selected from hydrogen and (1-4C)alkyl, and wherein Rₐ, R_{b}, R_{c}, R_{d}, Rₑ, R_{f}, R_{g} and Rₕ are each independently selected from hydrogen and (1-4C)alkyl.

6. The cosmetic or personal care composition as claimed in claim 3, wherein Y₁ is a group O⁻X₁ and Y₂ is a group O—R₂,
wherein R₂ is hydrogen; and optionally
wherein Y₂ is a group O⁻X₂ and Y₁ is a group O—R₁,
wherein R₁ is hydrogen.

7. The cosmetic or personal care composition as claimed in claim 3, wherein the monomeric moieties formed from the polymerisation of itaconic acid salts each independently have a structure according to formula (I) shown below: 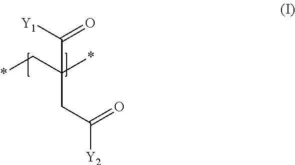
wherein
Y₁ is selected from:
(i) a group O⁻X₁,
(ii) a group O—R₁, wherein R₁ is hydrogen;
Y₂ is selected from:
(i) a group O⁻X₂,
(ii) a group O—R₂, wherein R₂ is hydrogen,
and the monomeric moieties formed from the polymerisation of itaconic acid and/or esters of itaconic acid each independently have a structure according to formula (II) shown below: 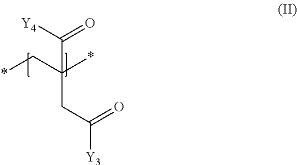
wherein
Y₃ is a group O—R₃, and
Y₄ is a group O—R₄;
with the proviso that at least one or Y₁ and Y₂ is a group O⁻X₁ or O⁻X₂.

8. The cosmetic or personal care composition as claimed in claim 3, wherein Y₁ is a group O⁻X₁ and Y₂ is a group O⁻X₂.

9. The cosmetic or personal care composition as claimed in claim 1, wherein the polymer consists essentially of monomeric moieties formed from the polymerisation of one or more of itaconic acid, sodium itaconate, potassium itaconate, ammonium itaconate, itaconic acid monomethyl ester, itaconic acid dimethyl ester, itaconic acid monoethyl ester and itaconic acid diethyl ester.

10. The cosmetic or personal care composition as claimed in claim 1, wherein the polymer comprises 50-100% of monomeric moieties formed from the polymerisation of itaconic acid salts.

11. The cosmetic or personal care composition as claimed in claim 1, wherein the polymer is:
e) a homopolymer formed from the polymerisation of sodium itaconate, or
f) a copolymer formed from the polymerisation of sodium itaconate and itaconic acid.

12. The cosmetic or personal care composition as claimed in claim 1, wherein the polymer has a degree of neutralisation of 50-100%.

13. Use of a polymer as claimed in claim 1, wherein the polymer has a molecular weight of 500-50000 Da.

14. The cosmetic or personal care composition as claimed in claim 1, wherein the polymer has a molecular weight of 8000-15000 Da.

15. The cosmetic or personal care composition as claimed in claim 1, wherein the cosmetic or personal care composition is for application to the skin, hair or nails.

16. The cosmetic or personal care composition as claimed in claim 1, wherein the composition has a pH of 4-8.

17. The cosmetic or personal care composition as claimed in claim 1, wherein the composition comprises 1-40 wt. % of a polymer defined in any preceding claim.

18. A process for the preparation of a cosmetic or personal care composition as claimed in claim 1, the process comprising the steps of:
g) providing a monomer mixture comprising water and either:
i. one or more salts of itaconic acid,
ii. a base, to which itaconic acid is subsequently added, or
iii. itaconic acid, to which a base is subsequently added;
h) reacting the mixture of step a) with a polymerisation initiator;
i) isolating the polymeric product resulting from step b); and
j) mixing the isolated polymeric product of step c) with one or more cosmetically acceptable diluents, excipients or carriers.

19. The process of claim 18, wherein step b) comprises reacting the mixture of step a) with a polymerisation initiator at a temperature of 50-100° C.; and optionally
wherein the quantity of initiator used in step b) is 1-50 w/w % with respect to the total quantity of monomers present in step a).

20. A method for forming a film on an external surface of the human body, the method comprising the step of:
k) applying to the external surface, a polymer or a cosmetic or personal care composition as claimed in claim 1;
wherein the amount of polymer or cosmetic or personal care composition applied to the external surface is sufficient to form a film on the external surface.

21. The method of claim 20, wherein the external surface is selected from one or more of the skin, hair and nails.
